(19) United States
(12) Patent Application Publication
(10) Pub. No.: US 2025/0387382 A1
(43) Pub. Date: Dec. 25, 2025

(54) METHODS OF TREATING ESSENTIAL TREMOR

(71) Applicant: Praxis Precision Medicines, Inc., Boston, MA (US)

(72) Inventor: Gabriel Maurice Belfort, Cambridge, MA (US)

(21) Appl. No.: 19/312,146

(22) Filed: Aug. 27, 2025

Related U.S. Application Data
(63) Continuation of application No. PCT/US2024/018009, filed on Mar. 1, 2024.
(60) Provisional application No. 63/449,528, filed on Mar. 2, 2023.
(60) Provisional application No. 63/449,676, filed on Mar. 3, 2023.
(60) Provisional application No. 63/467,471, filed on May 18, 2023.
(60) Provisional application No. 63/531,467, filed on Aug. 8, 2023.
(60) Provisional application No. 63/542,053, filed on Oct. 2, 2023.

(51) Int. Cl. A61K 31/445; A61P 25/14

(52) U.S. Cl. CPC A61K 31/445; A61P 25/14

(57) ABSTRACT

The present disclosure relates to methods of treating essential tremor that comprise administering to a subject in need thereof compound of formula (I):

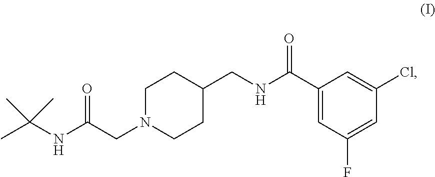 [SMILES: CC(C)(C)NC(=O)CN1CCC(CNC(=O)c2cc(F)cc(Cl)c2)CC1]

or a pharmaceutically acceptable salt thereof, wherein the compound of formula (I), or a pharmaceutically acceptable salt thereof, is administered to the subject once daily at a dose of about 60 mg to about 100 mg; such that The Essential Tremor Rating Assessment Scale (TETRAS) performance score of said subject is decreased following administration of the compound of formula (I), or a pharmaceutically acceptable salt thereof, as compared to the TETRAS performance score of said subject prior to the administration of the compound of formula (I), or a pharmaceutically acceptable salt thereof.

### RELATED APPLICATIONS

This application is a continuation of International Patent Application No. PCT/US2024/018009, filed on Mar. 1, 2024; which claims priority to U.S. Provisional Patent Application No. 63/542,053, filed on Oct. 2, 2023; U.S. Provisional Patent Application No. 63/531,467, filed on Aug. 8, 2023; U.S. Provisional Patent Application No. 63/467,471, filed on May 18, 2023; U.S. Provisional Patent Application No. 63/449,676, filed on Mar. 3, 2023; and U.S. Provisional Patent Application No. 63/449,528, filed on Mar. 2, 2023. The entire contents of each of the forgoing applications are hereby incorporated herein by reference.

### BACKGROUND

T-type calcium channels are low-voltage activated ion channels that mediate the influx of calcium into cells. Aberrant function or activity of these ion channels is associated with several diseases or conditions, including psychiatric disorders (e.g., mood disorders, such as a major depressive disorder), pain, tremor (e.g., essential tremor), epilepsy, or an epilepsy syndrome (e.g., absence seizures and juvenile myoclonic epilepsy).

In treating diseases or conditions related to aberrant function or activity of T-type calcium channels, the tolerability of potential pharmaceutical therapeutics is a factor to be considered. In investigations of new therapeutics, tolerability is a clinically-defined term that pertains to both patient (or study participant) and clinician (or investigator) input. The patient input includes their willingness to continue taking the therapeutic, and the clinician input includes their assessment of the severity and/or number of adverse effects associated with the therapeutic and a recommendation as to whether the patient should continue taking the therapeutic. Either party may decide that the drug is intolerable and thereby discontinue dosing.

Assessment of tolerability may also be based on the relationship between the adverse effects informing tolerability and the expected or perceived benefit. Accordingly, for a therapeutic or a therapeutic dose used for treating life-threatening diseases or conditions, more severe or an increased number of adverse events may be allowed before the therapeutic or the therapeutic dose may be designated as intolerable, as compared with a therapeutic or a therapeutic dose used for treating less serious diseases or conditions.

Essential tremor is a disorder with underlying aberrant T-type calcium channel function or activity, which may affect an individual's ability to function in daily life, but is typically not life-threatening. Essential tremor may, however, have a significant impact, on home life, work life, social life, or any combination thereof. For example, individuals with essential tremor may have difficulty performing daily activities, including certain tasks or jobs requiring motor skills, or feel embarrassed about their tremor. Essential tremor is one of the most common movement disorders, and is characterized by postural and/or kinetic tremors and, in some cases, impaired walking, along with some nonmotor symptoms. Propranolol (a beta blocker) and primidone (an anti-seizure medication), which are currently used for treating essential tremor, are characterized by serious side effects and are not effective in some patients. Accordingly, further methods of treating essential tremor are needed.

### SUMMARY

Accordingly, in some aspects, the present disclosure provides methods of treating essential tremor which comprise administering to a subject in need thereof, e.g., a human subject with essential tremor, a compound of formula (I):

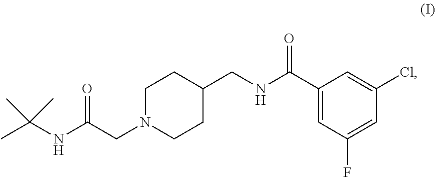 [SMILES: CC(C)(C)NC(=O)CN1CCC(CNC(=O)c2cc(F)cc(Cl)c2)CC1]

or a pharmaceutically acceptable salt thereof, wherein the compound of formula (I), or the pharmaceutically acceptable salt thereof, is administered to the subject once daily at a dose of about 60 mg to about 100 mg; such that The Essential Tremor Rating Assessment Scale (TETRAS) performance score of the subject is decreased following administration of the compound of formula (I), as compared to the TETRAS performance score of the subject prior to the administration of the compound of formula (I).

In some embodiments, the compound of Formula (I), or a pharmaceutically acceptable salt thereof, is administered to the subject at a dose of about 60 mg.

In some embodiments, the compound of formula (I), or a pharmaceutically acceptable salt thereof, is administered to the subject at a dose of about 100 mg.

In some embodiments, the compound of formula (I), or a pharmaceutically acceptable salt thereof, is administered to the subject following an initial titration period.

In some embodiments, the compound of formula (I), or a pharmaceutically acceptable salt thereof, is administered to the subject at a dose of about 60 mg following an initial titration period; wherein said initial titration period comprises administering to the subject the compound of formula (I), or a pharmaceutically acceptable salt thereof, once daily at a dose of about 5 mg during a first time period; followed by a dose of about 10 mg during a second time period; followed by a dose of about 20 mg during a third time period; followed by a dose of about 40 mg during a fourth time period.

In some embodiments, the first time period, the second time period, the third time period and the fourth time period are each 7 days long.

In some embodiments, the compound of formula (I), or a pharmaceutically acceptable salt thereof, is administered to said subject at a dose of about 100 mg following an initial titration period; wherein the initial titration period comprises administering to the subject the compound of formula (I), or a pharmaceutically acceptable salt thereof, once daily at a dose of about 5 mg during a first time period; followed by a dose of about 10 mg during a second time period; followed by a dose of about 20 mg during a third time period; followed by a dose of about 40 mg during a fourth time period; followed by a dose of about 60 mg during a fifth time period, followed by a dose of about 80 mg during a sixth time period.

In some embodiments, the first time period, the second time period, the third time period, the fourth time period, the fifth time period and the sixth time period are each 7 days long.

In some embodiments, the compound of formula (I), or a pharmaceutically acceptable salt thereof, is administered to the subject for at least 8 weeks.

In some embodiments, the compound of formula (I), or a pharmaceutically acceptable salt thereof, is administered to the subject for at least 12 weeks.

In some embodiments, the compound of formula (I), or a pharmaceutically acceptable salt thereof, is administered to the subject for at least 14 weeks.

In some embodiments, the compound of formula (I), or a pharmaceutically acceptable salt thereof, is administered to the subject for at least 52 weeks.

In some embodiments, the TETRAS performance score comprises ADL subscore, and wherein the ADL subscore is decreased by at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points following administration of the compound of formula (I), as compared to the ADL subscore of the subject prior to the administration of the compound of formula (I).

In some embodiments, the ADL subscore is decreased by at least about 2 points following at least 8 weeks of administration of the compound of formula (I), or a pharmaceutically acceptable salt thereof.

In some embodiments, the ADL subscore is decreased by at least about 3 points following at least 8 weeks of administration of the compound of formula (I), or a pharmaceutically acceptable salt thereof.

In some embodiments, the TETRAS performance score comprises mADL subscore, and wherein the mADL subscore is decreased by at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points following administration of the compound of formula (I), as compared to the ADL subscore of said subject prior to the administration of the compound of formula (I).

In some embodiments, the mADL subscore is decreased by at least about 2 points following at least 8 weeks of administration of the compound of formula (I), or a pharmaceutically acceptable salt thereof.

In some embodiments, the mADL subscore is decreased by at least about 3 points following at least 8 weeks of administration of the compound of formula (I), or a pharmaceutically acceptable salt thereof.

In some embodiments, the mADL subscore is decreased by at least about 4 points following at least 8 weeks of administration of the compound of formula (I), or a pharmaceutically acceptable salt thereof.

In some embodiments, a subscore for at least one item assessed as a part of mADL subscale is decreased, wherein said item is selected from the group consisting of: item 3 (drinking from a glass); item 4 (hygiene); item 5 (dressing); item 6 (pouring); item 7 (carrying); item 8 (using keys); item 9 (writing); and item 11 (overall disability).

In some embodiments, the TETRAS performance score comprises mADL11 subscore, and wherein the mADL11 subscore is decreased by at least about 0.5 points, at least about 1 point, at least about 2 points, at least about 3 points, at least about 4 points, at least about 5 points, at least about 6 points, at least about 7 points, at least about 8 points, at least about 9 points or at least about 10 points following administration of the compound of formula (I), as compared to the ADL subscore of said subject prior to the administration of the compound of formula (I).

In some embodiments, the mADL11 subscore is decreased by at least about 2 points following at least 8 weeks of administration of the compound of formula (I), or a pharmaceutically acceptable salt thereof.

In some embodiments, the mADL11 subscore is decreased by at least about 2.5 points following at least 8 weeks of administration of the compound of formula (I), or a pharmaceutically acceptable salt thereof.

In some embodiments, the subject does not have intention tremor.

In some embodiments, the subject is a human.

### BRIEF DESCRIPTION OF THE FIGURES

FIG. 1 is a schematic illustrating the design of the Phase 2 trial to evaluate the efficacy and safety/tolerability of titration of Compound 1 to 60 mg or 100 mg compared to placebo in participants with moderate to severe essential tremor (ET).

FIG. 2 is a schematic listing items that are included in the TETRAS and TETRAS modified total score.

FIG. 3 is a schematic showing the patient disposition in the clinical trial described in Example 1.

FIG. 4 is a table showing patient demographics and baseline characteristics (mITT).

FIG. 5 is a table showing TEASs experienced by the patients in the clinical trial described in Example 1.

FIG. 6 is a table showing TEAEs and the dose at onset of event.

FIG. 7 is a table showing discontinuations of Compound 1 in the mITT patient population.

FIG. 8 is a bar graph showing change in mADL and ADL scores for mITT population treated with Compound 1 (ulixacaltamide) and placebo.

FIG. 9 is a bar graph showing change in mADL score at day 56 for patients who took ulixacaltamide (Compound 1) or placebo.

FIG. 10 is a schematic showing observed change in scores for individual items that were evaluated as a part of mADL and ADL assessment as compared to baseline.

FIG. 11 is a bar graph showing fold-change adjusted by placebo change at day 56 for individual items that were evaluated as a part of mADL assessment.

FIG. 12 shows graphs illustrating the status of patients in the mITT population measured using PGI-C scale and CGI-S scale.

FIG. 13 is a schematic showing the results of the Pearson's correlation assessment for the change from baseline in the ADL and PS scores and other measures.

FIG. 14 is a bar graph showing change in mADL score at day 56 for each patient who took ulixacaltamide or placebo, when PS items (spirals—left and right, and handwriting) are excluded from assessment. The data from FIG. 8 is also included in FIG. 14 for comparison.

FIG. 15 is a graph showing change in mADL scores and mADL excluding PS scores (mADL11) at each scored timepoint. FIG. 15 shows improvement in both mADL scores and mADL excluding PS (mADL11) scores for ulixacaltamide vs. placebo after day 14.

FIG. 16 is a graph and a table showing endpoints analysis for mITT population.

FIG. 17 is a bar graph showing the results of post-hoc responder analysis using MCID Distribution Method in mITT population.

FIG. 18 is a bar graph showing mADL scores and mADL scores excluding PS in mITT population, excluding patients with intention tremor.

FIG. 19 is a schematic showing observed change in scores for individual items that are evaluated as a part of mADL assessment as compared to baseline in patients who do not have intention tremor.

FIG. 20 is a bar graph showing change in placebo-adjusted mADL scores excluding PS in mITT patients without intention tremor for the two tested dosing regiments (60 mg and 100 mg).

FIG. 21 is a bar graph showing change in mADL11 scores from baseline, by study week from week 8 to week 14.

FIG. 22 is a schematic showing the design of the Phase 3 trial to compare the efficacy and safety of ulixacaltamide (Compound 1) with placebo in participants aged 18 to 80 years who have a diagnosis of ET.

### DETAILED DESCRIPTION

### Methods of Treating Essential Tremor

In some aspects, the present disclosure provides methods of treating essential tremor that comprise administering to a subject in need thereof compound of formula (I):

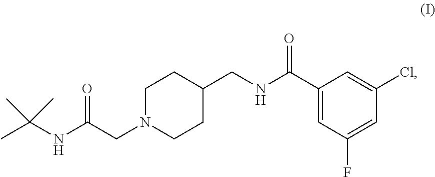 [SMILES: CC(C)(C)NC(=O)CN1CCC(CNC(=O)c2cc(F)cc(Cl)c2)CC1]

or a pharmaceutically acceptable salt thereof, wherein the compound of formula (I), or the pharmaceutically acceptable salt thereof, is administered to the subject once daily at a dose of about 60 mg to about 100 mg; such that The Essential Tremor Rating Assessment Scale (TETRAS) performance score of the subject is decreased following administration of the compound of formula (I), as compared to the TETRAS performance score of the subject prior to the administration of the compound of formula (I).

As used herein, a “subject” to which administration is contemplated includes, but is not limited to, a human and a non-human animal. A non-human animal may be a mammal, such as a primate (e.g., a cynomolgus monkey or a rhesus monkey), cattle, a pig, a horse, a sheep, a goat, a rodent (e.g., a mouse or a rat), a cat or a dog. In some embodiments, the subject is a non-human animal. In other embodiments, the subject is a human. In some embodiments, the subject is a male or female of any age group, such as a pediatric subject (e.g., infant, child, adolescent) or an adult subject (e.g., young adult, middle-aged adult or senior adult) The terms “human” and “patient” may be used interchangeably herein.

In some embodiments, the subject is a human. In some embodiments, the subject has essential tremor. In some embodiments, the subject does not have intention tremor.

As used herein, and unless otherwise specified, the terms “treat,” “treating” and “treatment” contemplate an action that occurs while a subject is suffering from the specified disease, disorder or condition, e.g., essential tremor, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

Methods of the present disclosure comprise administering to a subject in need thereof the compound of formula (I):

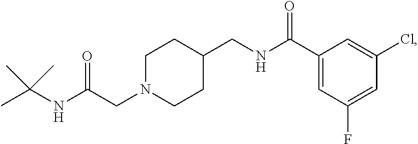 [SMILES: CC(C)(C)NC(=O)CN1CCC(CNC(=O)c2cc(F)cc(Cl)c2)CC1]

or a pharmaceutically acceptable salt (e.g., co-crystal) or solvate thereof.

As used herein, the term “pharmaceutically acceptable salt” refers to those salts which are, within sound medical judgment, suitable for use in contact with tissue of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compound of formula (I) include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include acetate, adipate, alginate, ascorbate, aspartate, benzenesulfonate, benzoate, besylate, bisulfate, borate, butyrate, camphorate, camphorsulfonate, citrate, cyclopentanepropionate, cyclamate, digluconate, dodecylsulfate, edisylate, ethanesulfonate, esylate, formate, fumarate, gentisate, glucoheptonate, glycerophosphate, gluconate, glucoronate, glutamate, glutarate, glycolate, hemisulfate, heptanoate, hexanoate, hippurate, hydroiodide, 2-hydroxy-ethanesulfonate, isethionate, ketoglutarate, lactobionate, lactate, laurate, lauryl sulfate, malate, maleate, malonate, mesylate, methanesulfonate, napadisylate, napsylate, 2-naphthalenesulfonate, nicotinate, nitrate, oleate, oxalate, oroate, palmitate, pamoate, pectinate, persulfate, 3-phenylpropionate, phosphate, picrate, pivalate, propionate, sebacate, stearate, succinate, sulfate, tartrate, thiocyanate, p-toluenesulfonate, undecanoate, valerate salts, stereoisomers thereof (e.g., enantiomers, diastereomers) and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

In certain embodiments, compound of formula (I) is a hydrochloric acid salt, for example, compound of formula (II):

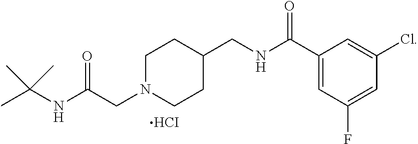

The compound of formula (I) may also be referred to as N-((1-(2-(tert-butylamino)-2-oxoethyl)piperidin-4-yl)methyl)-3-chloro-5-fluorobenzamide, while formula (II) may be referred to as N-((1-(2-(tert-butylamino)-2-oxoethyl)piperidin-4-yl)methyl)-3-chloro-5-fluorobenzamide hydrochloride.

In some embodiments, the compound of formula (I) or formula (II) may also be referred to herein as “ulixacaltamide” or “Compound 1”.

In some embodiments, methods of the present disclosure comprise administering to a subject in need thereof a crystalline form of the compound of formula (II), wherein the crystalline form exhibits an X-ray powder diffraction pattern comprising peaks at the following diffraction angles (2θ): 16.2±0.2, 17.4±0.2, and 26.6±0.2. In some embodiments, the crystalline form exhibits an X-ray powder diffraction pattern comprising peaks at the following diffraction angles (2θ): 11.5±0.2, 16.2±0.2, 17.4±0.2, 22.6±0.2, and 26.6±0.2. In some embodiments, the crystalline form exhibits an X-ray powder diffraction pattern comprising peaks at the following diffraction angles (2θ): 11.5±0.2, 16.2±0.2, 17.4±0.2, 18.3±0.2, 18.5±0.2, 19.2±0.2, 20.0±0.2, 22.6±0.2, 23.9±0.2, and 26.6±0.2. In some embodiments, the powder X-ray diffraction pattern was obtained using Cu Kα radiation. In some embodiments, the crystalline form has a melting point onset as determined by differential scanning calorimetry at about 226.6° C.

In some embodiments, methods of the present disclosure comprise administering to a subject in need thereof a crystalline form of the compound of formula (II), wherein the crystalline form exhibits an X-ray powder diffraction pattern comprising peaks at the following diffraction angles (2θ): 21.9±0.2, 18.5±0.2, and 17.8±0.2. In some embodiments, the crystalline form exhibits an X-ray powder diffraction pattern comprising peaks at the following diffraction angles (2θ): 21.9±0.2, 18.5±0.2, 17.8±0.2, 10.2±0.2, and 20.5±0.2. In some embodiments, the crystalline form exhibits an X-ray powder diffraction pattern comprising peaks at the following diffraction angles (2θ): 21.9±0.2, 18.5±0.2, 17.8±0.2, 10.2±0.2, 20.5±0.2, 25.2±0.2, 16.9±0.2, 24.2±0.2, 28.6±0.2, and 21.2±0.2. In some embodiments, the powder X-ray diffraction pattern was obtained using Cu Kα radiation. In certain embodiments, the crystalline form has a melting point onset as determined by differential scanning calorimetry at about 97.9, 131.6, 223.7, 83.8, 128.9, 168.9, or 224.4° C.

In some embodiments, compound of formula (I), compound of formula (II), or crystalline form of the compound of formula (II) is administered to a subject in need thereof in accordance with methods of the present disclosure as a part of a pharmaceutical composition also comprising a modified-release polymer. The term “modified-release polymer”, as used herein, refers to a polymer that is used in a composition (e.g., a tablet or a capsule) to modify the release rate of the drug upon administration to a subject. For example, a modified-release polymer is used to dissolve a drug over time in order to be released more slowly and steadily into the bloodstream. For example, a modified-release polymer is a controlled-release polymer. For example, a modified-release polymer or a controlled-release polymer is an HPMC polymer. In some embodiments, a modified-release polymer may include hydrophilic matrix polymers (e.g., hypromellose, HPMC (hydroxyl-propyl methylcellulose)), hydrophobic matrix polymers (e.g., ethyl cellulose, ethocel), or polyacrylate polymers (e.g., Eudragit RL100, Eudragit RS100).

In some embodiments, the pharmaceutical composition comprising the compound of formula (I), the compound of formula (II), or crystalline form of the compound of formula (II) comprises a diluent. The term “diluent”, as used herein, refers to an excipient used to increase weight and improve content uniformity. For example, diluents include cellulose derivatives (e.g., microcrystalline cellulose), starches (e.g., hydrolyzed starches, and partially pregelatinized starches), anhydrous lactose, lactose monohydrate, di-calcium phosphate (DCP), and sugar alcohols (e.g., sorbitol, xylitol and mannitol)).

In some embodiments, the pharmaceutical composition comprising the compound of formula (I), the compound of formula (II), or crystalline form of the compound of formula (II) comprises a glidant. The term “glidant” as used herein refers to an excipient used to promote powder flow by reducing interparticle friction and cohesion. For example, glidants include fumed silica (e.g., colloidal silicon dioxide), talc, and magnesium carbonate.

In some embodiments, the pharmaceutical composition comprising the compound of formula (I), the compound of formula (II), or crystalline form of the compound of formula (II) comprises a lubricant. The term “lubricant” as used herein refers to an excipient used to prevent ingredients from clumping together and from sticking to the tablet punches or capsule filling machine. Lubricants are also used to ensure that tablet formation and ejection can occur with low friction between the solid and die wall. For example, lubricants include magnesium stearate, calcium stearate, stearic acid, talc, silica, and fats (e.g., vegetable stearin).

In some embodiments, the pharmaceutical composition comprising the compound of formula (I), the compound of formula (II), or crystalline form of the compound of formula (II) comprises a coating. The term “coating” as used herein refers to an excipient, for example, to protect tablet ingredients from deterioration by moisture in the air and to make large or unpleasant-tasting tablets easier to swallow.

Methods of the present disclosure for treating essential tremor comprise administering to a subject in need thereof a titrated dose of the compound of formula (I), or a pharmaceutically acceptable salt thereof, such that the end or maintenance dosage (i.e., final dosage) exceeds an initial dosage or a dosage at which adverse events are likely to be experienced absent titration (a maximum tolerated dosage achieved without titration). As used herein, administering a tritrated dose refers to the practice of beginning with a low dosage and escalating to one or more higher dosages. Administration of titrated doses of the compound of formula (I), or a pharmaceutically acceptable salt thereof, is described, e.g., in WO 2021/222342 A1, the entire contents of which are hereby incorporated herein by reference.

In some embodiments, the compound of formula (I), or a pharmaceutically acceptable salt thereof, is administered to a subject in the context of the present disclosure at a final dose (e.g., 60 mg or 100 mg) after an initial titration period. The term “initial titration period”, when used in reference to administration of a final dose of the compound of formula (I), or a pharmaceutically acceptable salt thereof, e.g., a dose of about 60 mg or about 100 mg, refers to a period of time during which escalating doses which are lower than the final dose of the compound of formula (I), or a pharmaceutically acceptable salt thereof, are administered to a subject until the final dose of the compound of formula (I), or a pharmaceutically acceptable salt thereof, is achieved, e.g., a dose of about about 60 mg or about 100 mg.

For example, an initial titration period may comprise administration to a subject of escalating doses of the compound of formula (I), or a pharmaceutically acceptable salt thereof, until a dose of about 60 mg is achieved. In some embodiments, an initial titration period may comprise administration to a subject of one or more doses of the compound of formula (I), or a pharmaceutically acceptable salt thereof, selected from the group consisting of: a dose of about 5 mg, a dose of about 10 mg, a dose of about 20 mg and a dose of about 40 mg. In some embodiments, an initial titration period may comprise administration to a subject of a dose of the compound of formula (I), or a pharmaceutically acceptable salt thereof, of about 5 mg during a first time period; followed by a dose of about 10 mg during a second time period; followed by a dose of about 20 mg during a third time period; followed by a dose of about 40 mg during a fourth time period. In some embodiments, the first time period may be about 1 day, about 2 days, about 3 days, about 4 days, about 5 days, about 6 days or about 7 days long. In some embodiments, the second time period may be about 1 day, about 2 days, about 3 days, about 4 days, about 5 days, about 6 days or about 7 days long. In some embodiments, the third time period may be about 1 day, about 2 days, about 3 days, about 4 days, about 5 days, about 6 days or about 7 days long. In some embodiments, the fourth time period may be about 1 day, about 2 days, about 3 days, about 4 days, about 5 days, about 6 days or about 7 days long. In some embodiments, the first, second, third and fourth time periods are each 7 days long. In some embodiments, the compound of formula (I), or a pharmaceutically acceptable salt thereof, is administered once daily.

In another example, an initial titration period may comprise administration to a subject of escalating doses of the compound of formula (I), or a pharmaceutically acceptable salt thereof, until a dose of about 80 mg is achieved. In some embodiments, an initial titration period may comprise administration to a subject of one or more doses of the compound of formula (I), or a pharmaceutically acceptable salt thereof, selected from the group consisting of: a dose of about 5 mg, a dose of about 10 mg, a dose of about 20 mg, a dose of about 40 mg, and a dose of about 60 mg. In some embodiments, an initial titration period may comprise administration to a subject of a dose of the compound of formula (I), or a pharmaceutically acceptable salt thereof, of about 5 mg during a first time period; followed by a dose of about 10 mg during a second time period; followed by a dose of about 20 mg during a third time period; followed by a dose of about 40 mg during a fourth time period; followed by a dose of about 60 mg during the fifth time period. In some embodiments, the first time period may be about 1 day, about 2 days, about 3 days, about 4 days, about 5 days, about 6 days or about 7 days long. In some embodiments, the second time period may be about 1 day, about 2 days, about 3 days, about 4 days, about 5 days, about 6 days or about 7 days long. In some embodiments, the third time period may be about 1 day, about 2 days, about 3 days, about 4 days, about 5 days, about 6 days or about 7 days long. In some embodiments, the fourth time period may be about 1 day, about 2 days, about 3 days, about 4 days, about 5 days, about 6 days or about 7 days long. In some embodiments, the fifth time period may be about 1 day, about 2 days, about 3 days, about 4 days, about 5 days, about 6 days or about 7 days long. In some embodiments, the first, second, third, fourth and fifth time periods are each 7 days long. In some embodiments, the compound of formula (I), or a pharmaceutically acceptable salt thereof, is administered once daily.

In yet another example, an initial titration period may comprise administration to a subject of escalating doses of the compound of formula (I), or a pharmaceutically acceptable salt thereof, until a dose of about 100 mg is achieved. In some embodiments, an initial titration period may comprise administration to a subject of one or more doses of the compound of formula (I), or a pharmaceutically acceptable salt thereof, selected from the group consisting of: a dose of about 5 mg, a dose of about 10 mg, a dose of about 20 mg, a dose of about 40 mg, a dose of about 60 mg and a dose of about 80 mg. In some embodiments, an initial titration period may comprise administration to a subject of a dose of the compound of formula (I), or a pharmaceutically acceptable salt thereof, of about 5 mg during a first time period; followed by a dose of about 10 mg during a second time period; followed by a dose of about 20 mg during a third time period; followed by a dose of about 40 mg during a fourth time period; followed by a dose of about 60 mg during the fifth time period; followed by a dose of about 80 mg during the sixth time period. In some embodiments, the first time period may be about 1 day, about 2 days, about 3 days, about 4 days, about 5 days, about 6 days or about 7 days long. In some embodiments, the second time period may be about 1 day, about 2 days, about 3 days, about 4 days, about 5 days, about 6 days or about 7 days long. In some embodiments, the third time period may be about 1 day, about 2 days, about 3 days, about 4 days, about 5 days, about 6 days or about 7 days long. In some embodiments, the fourth time period may be about 1 day, about 2 days, about 3 days, about 4 days, about 5 days, about 6 days or about 7 days long. In some embodiments, the fifth time period may be about 1 day, about 2 days, about 3 days, about 4 days, about 5 days, about 6 days or about 7 days long. In some embodiments, the sixth time period may be about 1 day, about 2 days, about 3 days, about 4 days, about 5 days, about 6 days or about 7 days long. In some embodiments, the first, second, third, fourth, fifth and sixth time periods are each 7 days long. In some embodiments, the compound of formula (I), or a pharmaceutically acceptable salt thereof, is administered once daily.

In certain aspects of all embodiments disclosed herein, the time period of administration, such as the first, second, third, fourth, fifth, or sixth time period, may range from about 3 days to about 1 year. For example, the first, second, third, fourth, fifth and/or sixth time period may be 3 days, 4 days, 5 days, 6 days, 7 days, 8 days, 9 days, 10 days, 11 days, 12 days, 13 days, 14 days, 15 days, 16 days, 21 days, 28 days, 35 days, 42 days, 49 days, 56 days, 63 days, 70 days, 77 days, 84 days, 91 days, 98 days, 105 days, 112 days, 119 days, 126 days, 133 days, 140 days, 147 days, 154 days, 161 days, 168 days, 175 days, 182 days, 189 days, 196 days, 203 days, 210 days, 217 days, 224 days, 231 days, 238 days, 245 days, 252 days, 259 days, 266 days, 273 days, 280 days, 287 days, 294 days, 301 days, 308 days, 315 days, 322 days, 329 days, 336 days, 343 days, 350 days, 357 days, 364 days, or 1 year long. In certain aspects of the embodiments disclosed herein, the time period of administration, such as the seventh time period, may range from about 3 to about 16 days, such as, for example, 3, 4, 5, 6, 7, 8, 9, 10, 11, 12, 13, 14, 15, or 16 days. In other aspects of the embodiments disclosed herein, of the time periods, e.g., the first, second, third, fourth, fifth, sixth, or seventh time period of administration may be greater than 14 days.

In certain aspects, the dosage increase relative to the prior dose is not more than 40 mg per day. For instance, in certain embodiments, the second dose is increased no more than 40 mg per day relative to the first dose, and in certain aspects, the third dose is increased no more than 40 mg per day relative to the second dose. In other aspects, the dosage increase relative to the prior dose is not more than 20 mg per day. For instance, in certain embodiments, the second dose is increased no more than 20 mg per day relative to the first dose, and in certain embodiments, the third dose is increased no more than 20 mg per day relative to the second dose. In some embodiments, the third dose is the maintenance, or final dose.

In the context of the present disclosure, a compound of formula (I), or a pharmaceutically acceptable salt thereof, may be administered to a subject once, twice, three times daily at a dose of up to about 120 mg (e.g., from about 5 mg to about 120 mg, from about 10 mg to about 120 mg, from about 15 mg to about 120 mg, from about 20 mg to about 120 mg, from about 40 mg to about 120 mg, from about 5 mg to about 100 mg, from about 10 mg to about 100 mg, from about 15 mg to about 100 mg, from about 20 mg to about 100 mg, from about 40 mg to about 100 mg, from about 5 mg to about 80 mg, from about 10 mg to about 80 mg, from about 15 mg to about 80 mg, from about 20 mg to about 80 mg, from about 40 mg to about 80 mg, from about 5 mg to about 60 mg, from about 10 mg to about 60 mg, from about 15 mg to about 60 mg, from about 20 mg to about 60 mg, or from about 40 mg to about 60 mg). In some embodiments, the compound of formula (I), or a pharmaceutically acceptable salt thereof, is administered to the subject once daily.

In some embodiments, methods of treating essential tremor in a subject in need thereof in the context of the present disclosure comprise:

(a) administering to the subject once daily during a first time period (e.g., 3, 4, 5, 6, 7, 8, or 9 days), 5 mg of the compound of formula (I) or a pharmaceutically acceptable salt thereof (e.g., the compound of formula (II));

(b) administering to the subject once daily during a second time period (e.g., 3, 4, 5, 6, 7, 8, or 9 days), 10 mg of the compound of formula (I) or a pharmaceutically acceptable salt thereof (e.g., the compound of formula (II)); and

(c) administering to the subject once daily during a third time period (e.g., 3, 4, 5, 6, 7, 8, or 9 days), 20 mg of the compound of formula (I) or a pharmaceutically acceptable salt thereof (e.g., the compound of formula (II)). In some embodiments, a compound of formula (I), or a pharmaceutically acceptable salt thereof, is administered to the subject once daily.

In some embodiments, methods of treating essential tremor in a subject in need thereof in the context of the present disclosure comprise:

(a) administering to the subject during a first time period (e.g., 3, 5, 6, 7, 8, or 9 days), 20-40 mg per day of the compound of formula (I) or a pharmaceutically acceptable salt thereof (e.g., the compound of formula (II));

(b) administering to the subject during a second time period (e.g., 3, 5, 6, 7, 8, or 9 days), 20-60 mg per day of the compound of formula (I) or a pharmaceutically acceptable salt thereof (e.g., the compound of formula (II)); and

(c) administering to the subject during a third period (e.g., 3, 4, 5, 6, 7, 8, or 9 days), 20-80 mg per day of the compound of formula (I) or a pharmaceutically acceptable salt thereof (e.g., the compound of formula (II)).

In some embodiments, the method further comprises (d) administering to the subject during a fourth time period (e.g., 3, 4, 5, 6, 7, 8, or 9 days), 20-100 mg per day of the compound of formula (I) or a pharmaceutically acceptable salt thereof (e.g., the compound of formula (II)).

In other embodiments, the method further comprises (e) administering to the subject during a fifth time period (e.g., 3, 4, 5, 6, 7, 8, or 9 days), 20-120 mg per day of the compound of formula (I) or a pharmaceutically acceptable salt thereof (e.g., the compound of formula (II)).

In certain embodiments, the method further comprises (f) administering to the subject during a sixth time period (e.g., 3, 4, 5, 6, 7, 8, 9, 10, 11, 12, 13, 14, 15, or 16 days), 20-120 mg per day of the compound of formula (I) or a pharmaceutically acceptable salt thereof (e.g., the compound of formula (II)).

In certain embodiments, the method further comprises (g) administering to the subject during a seventh time period (e.g., 3, 4, 5, 6, 7, 8, 9, 10, 11, 12, 13, or 14 or more days), 20-120 mg per day of the compound of formula (I) or a pharmaceutically acceptable salt thereof (e.g., the compound of formula (II)) as needed.

A physician may elect not to continue to escalate the dose of the compound of formula (I) or a pharmaceutically acceptable salt thereof, such that there may be less than the seven time periods described above. For example, there may be only 1, 2, 3, 4, 5, or 6 time periods of escalating dosages needed to achieve a desired thereapeutic effect. In some embodiments, methods of the present disclosure comprise 2, 3 or 4 time periods of escalating dosages. In some embodiments, methods of the present disclosure comprise 5 time periods of escalating dosages. In some embodiments, methods of the present disclosure comprise 7 time periods of escalating dosages.

In some embodiments, methods of the present disclosure comprise: (a) administering to the subject during a first time period ranging from about 3 to about 9 days, about 5 mg to about 40 mg per day of a compound of formula (I) or a pharmaceutically acceptable salt thereof, such as 20 mg or 40 mg per day; (b) administering to the subject during a second time period ranging from about 3 to about 9 days, about 10 mg to about 100 mg per day of the compound of formula (I) or a pharmaceutically acceptable salt thereof, such as 40 mg, 60 mg, or 80 mg per day; and (c) administering to the subject during a third time period ranging from about 3 to about 9 days, about 20 mg to about 120 mg per day of the compound of formula (I) or a pharmaceutically acceptable salt thereof, such as 60 mg, 80 mg, 100 mg, or 120 mg per day.

In some embodiments, methods of the present disclosure comprise: (a) administering a first dose of about 20 mg to about 40 mg per day of a compound of formula (I) or a pharmaceutically acceptable salt thereof to a subject once daily during a first time period; (b) increasing the amount of the compound in the first dose and administering one or more increased doses of the compound to the subject to arrive at a maximum titrated dose of about 80 mg to about 120 mg per day; and (c) administering the maximum titrated dose to the subject once daily as needed.

In related embodiments, the dosage of the compound of formula (I) may be adjusted upward or downward in 1, 2, 3, 4, 5, 10, 15, 20 mg increments as deemed necessary by a physician depending on a subject's response to the prior dosages of compound of formula (I).

In various embodiments of the disclosure, methods disclosed herein comprise: (a) administering a first dose of 20 mg per day during a first time period of 3 days, (b) administering a second dose of 40 mg per day during a second time period 3 days, (c) administering a third dose of 60 mg per day during a third time period of 7 days; (d) administering a fourth dose of 80 mg per day during a fourth time period of 7 days; (c) administering a fifth dose of 100 mg per day during a fifth time period of 7 days; and (f) thereafter administering a sixth dose of 120 mg per day as needed. In various other embodiments, the methods disclosed herein comprise (a) administering a first dose of 20 mg per day for a first time period of 3 days, (b) administering a second dose of 40 mg per day for a second time period of 3 days, (c) administering a third dose of 80 mg per day for a third time period of 3 days; and (d) administering a fourth dose of 120 mg per day as needed.

In certain embodiments there is disclosed a method of treating a disease or condition relating to aberrant function or activity of T-type calcium channels in a subject in need thereof comprising (a) administering to the subject during a first time period of 3 days about 20 mg per day of a compound of formula (I) or a pharmaceutically acceptable salt thereof; (b) administering to the subject during a second time period of about 3 days about 40 mg per day of the compound; (c) administering to the subject during a third time period of about 3 days about 60 mg per day of the compound; (d) administering to the subject during a fourth time period of about 3 days about 80 mg per day of the compound; (c) administering to the subject during a fifth time period of about 3 days about 100 mg per day of the compound; and (f) administering to the subject during a sixth time period of time about 120 mg per day of the compound. Instead of every 3 days, the doses can also be administered every 4, 5, or 6 days.

Also disclosed herein is a method of treating essential tremor in a subject in need thereof, comprising: (a) administering to the subject during a first time period of 7 days about 20 mg per day of a compound of formula (I) or a pharmaceutically acceptable salt thereof; (b) administering to the subject during a second time period of about 7 days about 40 mg per day of the compound; (c) administering to the subject during a third time period of about 7 days about 60 mg per day of the compound; (d) administering to the subject during a fourth time period of about 7 days about 80 mg per day of the compound; (c) administering to the subject during a fifth time period of about 7 days about 100 mg per day of the compound; and (f) administering to the subject during a sixth period of time about 120 mg per day of the compound.

In some embodiment, methods of the present disclosure comprise: (a) administering to the subject during a first time period of 3 days about 40 mg per day of a compound of formula (I) or a pharmaceutically acceptable salt thereof; (b) administering to the subject during a second time period of about 3 days about 80 mg per day of the compound; and (c) administering to the subject during a third period about 120 mg per day of the compound. Instead of every 3 days, the doses can also be administered every 4, 5, or 6 days.

Also disclosed herein is a method of treating a disease or condition relating to aberrant function or activity of a T-type calcium channel in a subject in need thereof comprising (a) administering to the subject for a first period of 7 days about 40 mg per day of a compound of formula (I) or a pharmaceutically acceptable salt thereof; (b) administering to the subject for a second period of about 7 days about 80 mg per day of the compound; and (c) administering to the subject for a third period about 120 mg per day of the compound.

In certain embodiments there is disclosed a method of treating a disease or condition relating to aberrant function or activity of a T-type calcium channel in a subject in need thereof comprising (a) administering to the subject for a first period of 7 days about 20 mg per day of a compound of formula (I) or a pharmaceutically acceptable salt thereof; (b) administering to the subject for a second period of about 7 days about 40 mg per day of the compound; (c) administering to the subject for a third period of about 7 days about 60 mg per day of the compound; (d) administering to the subject for a fourth period of about 7 days about 80 mg per day of the compound; (c) administering to the subject for a fifth period of about 7 days about 100 mg per day of the compound; (f) administering to the subject for a sixth period of about 14 days about 120 mg per day of the compound; and (g) thereafter administering to the subject about 1-120 mg per day of the compound as needed.

A physician may choose to stop escalating the dose of compound of formula (I) or a pharmaceutically acceptable salt thereof once the subject has demonstrated a desired therapeutic effect. At this point, the physician may elect for the subject to continue the dose that the subject has been taking to achieve the desired therapeutic effect, or may elect for the subject to lower the dose of the compound of formula (I), or a pharmaceutically acceptable salt thereof, in order to maintain the desired thereapeutic effect, reduce side effects, or both. Achieving a desired therapeutic effect may be observed by improvements in, for example, the subject's TETRAS performance score, accelerometer performance score, or Archimedes spiral task test.

In some embodiments, methods of treating essential tremor provided by the present disclosure comprise administering to a subject in need thereof a compound of formula (I), or a pharmaceutically acceptable salt thereof, such that The Essential Tremor Rating Assessment Scale (TETRAS) performance score of the subject is decreased following administration of the compound of formula (I), or a pharmaceutically acceptable salt thereof, as compared to the TETRAS performance score of the subject prior to the administration of the compound of formula (I), or a pharmaceutically acceptable salt thereof. TETRAS was developed to quantify essential tremor severity and its impact on activities of daily living, as described in Elble et al., J. Neurol Neuromedicine (2016), 1(4):34-38, the entire contents of which are hereby incorporated herein by reference. In certain embodiments, methods of treating essential tremor in a subject in need thereof, may result in a change in TETRAS performance score of the subject. The TETRAS performance score is assessed using the TETRAS scale, which consists of two sections: the Performance Subscale (TETRAS-PS) and the Activities of Daily Living (TETRAS-ADL) subscale. TETRAS-PS section includes 9 items as listed below:

| Item No. | Item Description |
|---|---|
| 1 | Head tremor |
| 2 | Face tremor |
| 3 | Voice tremor |
| 4 | Kinetic tremor |
| 5 | Lower limb tremor |
| 6 | Spiral drawing |
| 7 | Handwriting |
| 8 | Dot approximation task |
| 9 | Standing |

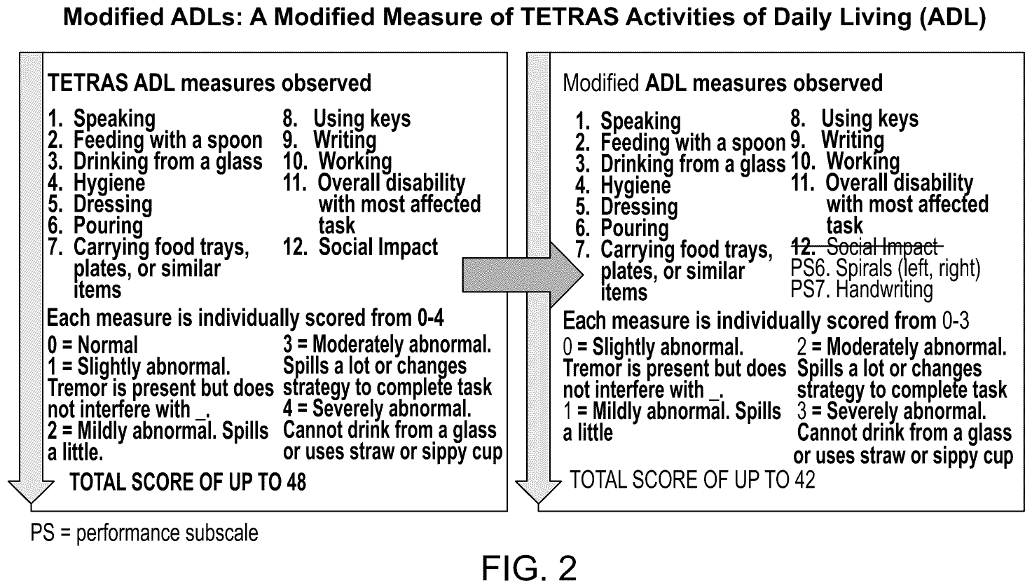

The TETRAS-ADL, or ADL subscale is a 12-item assessment of typical daily activities that are impacted by tremor. As shown in FIG. 2, activities in the TETRAS-ADL subscale are assessed in the following functional domains:

| Item No. | Item Description |
|---|---|
| 1 | Speaking |
| 2 | Feeding (e.g., with a spoon) |
| 3 | Drinking from a glass |
| 4 | Hygene (e.g., personal hygiene) |
| 5 | Dressing |
| 6 | Pouring |
| 7 | Carrying (e.g., food trays, plates or similar) |
| 8 | Using keys |
| 9 | Writing |
| 10 | Working |
| 11 | Overall disability with most affected task |
| 12 | Social impact |

The impact to each function is rated on a 5-point Likert scale from 0 to 4. The full ADL subscale score is calculated as the sum of all 12 items and ranges from 0 to 48.

TETRAS modified ADL subscore (mADL) is a a composite sum of items 1 to 11 in TETRAS-ADL subscale (omitting item 12 social impact) and items 6 and 7 of the TETRAS-PS subscale (spiral drawing and handwriting). Scoring in mADL is adjusted, such that response options 0 and 1 are combined into a single response of “0=Normal.”, and scores of 2, 3, and 4 are adjusted to 1, 2, and 3, respectively. The TETRAS modified total score is the sum of the adjusted TETRAS-ADL and TETRAS PS items, having a possible range of 0 to 42, where larger values represent increased direct tremor impact to activities of daily living. The items that are included in the TETRAS-modified total score are illustrated in FIG. 2.

TETRAS modified ADL excluding performance subscale (mADL11) score is a composite sum of items 1 to 11 of the TETRAS-ADL subscale (omitting item 12 social impact score and items 6 and 7 of TETRAS-PS subscale). Scoring of each item is transformed to a 4-point scale from 0 to 3 with total score range from 0 to 33.

In some embodiments, a decrease in TETRAS performance score, e.g., one or more of ADL, mADL or mADL11, is indicative of a desired therapeutic effect. In some embodiments, a decrease in TETRAS performance score, e.g., one or more of ADL, mADL or mADL11, is indicative of an improvement in the symptoms of essential tremor.

In some embodiments, methods of treating essential tremor in a subject in need thereof provided by the present disclosure may result in a decrease in TETRAS performance score in the subject. Thus, in some embodiments, TETRAS performance score of a subject is decreased following administration of the compound of formula (I), or a pharmaceutically acceptable salt, to the subject in accordance to methods of the present disclosure, as compared to TETRAS performance score of the subject prior to administration of the compound of formula (I), or a pharmaceutically acceptable salt thereof.

In some embodiments, the TETRAS performance score comprises ADL subscore, and the ADL subscore of a subject is decreased by at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points following administration of the compound of formula (I), as compared to the ADL subscore of the subject prior to the administration of the compound of formula (I). In some embodiments, the ADL subscore is decreased by at least about 2 points following at least 8 weeks of administration of the compound of formula (I), or a pharmaceutically acceptable salt thereof to the subject. In some embodiments, the ADL subscore is decreased by at least about 3 points following at least 8 weeks of administration of the compound of formula (I), or a pharmaceutically acceptable salt thereof to the subject.

In some embodiments, the TETRAS performance score comprises mADL subscore, and the mADL subscore of a subject is decreased by at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points following administration of the compound of formula (I), as compared to the mADL subscore of the subject prior to the administration of the compound of formula (I). In some embodiments, the mADL subscore is decreased by at least about 2 points following at least 8 weeks of administration of the compound of formula (I), or a pharmaceutically acceptable salt thereof to the subject. In some embodiments, the mADL subscore is decreased by at least about 3 points following at least 8 weeks of administration of the compound of formula (I), or a pharmaceutically acceptable salt thereof to the subject. In some embodiments, the mADL subscore is decreased by at least about 2 points following at least 8 weeks of administration of the compound of formula (I), or a pharmaceutically acceptable salt thereof, to the subject.

In some embodiments, in the context of the present disclosure, administration of the compound of formula (I), or a pharmaceutically acceptable salt thereof, to a subject in need thereof for treating essential tremor leads to a decrease in a subscore for at least one item assessed as a part of mADL subscale. In some embodiment, the item is selected from the group consisting of: item 3 (drinking from a glass); item 4 (hygiene); item 5 (dressing); item 6 (pouring); item 7 (carrying); item 8 (using keys); item 9 (writing); and item 11 (overall disability).

In some embodiments, the TETRAS performance score comprises mADL11 subscore, and the mADL11 subscore of a subject is decreased by at least about 0.5 points, at least about 1 point, at least about 2 points, at least about 3 points, at least about 4 points, at least about 5 points, at least about 6 points, at least about 7 points, at least about 8 points, at least about 9 points or at least about 10 points, following administration of the compound of formula (I), as compared to the mADL11 subscore of the subject prior to the administration of the compound of formula (I). In some embodiments, the mADL11 subscore is decreased by at least about 2 points following at least 8 weeks of administration of the compound of formula (I), or a pharmaceutically acceptable salt thereof. In some embodiments, mADL11 subscore is decreased by at least about 2.5 points following at least 8 weeks of administration of the compound of formula (I), or a pharmaceutically acceptable salt thereof.

In some embodiments, methods of treating a disease or condition relating to aberrant function or activity of a T-type calcium channel, e.g., essential tremor, in a subject in need thereof may result in a change, e.g., a decrease, in TETRAS performance score of the subject. In some embodiments, methods of treating a disease or condition relating to aberrant function or activity of a T-type calcium channel, e.g., essential tremor, in a subject in need thereof, may result in a change, e.g., a decrease, in ADL performance subscore score of the subject. In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore of the subject. In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore score of the subject of at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points. In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore score of the subject of 2 points or greater. In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore score of the subject of at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least 10 points when compound of formula (I) is administered at a dose of about 60 mg once daily after an initial titration period.

In some embodiments, the compound of formula (I), or a pharmaceutically acceptable salt thereof, is administered to a subject in need thereof in the context of the present disclosure for at least 8 weeks, at least 12 weeks, at least 14 weeks, at least 52 weeks, at least 2 years, at least 3 years, at least 4 years, at least 5 years or at least 10 years.

In some embodiments, methods of treating a disease or condition relating to aberrant function or activity of a T-type calcium channel, e.g., essential tremor, in a subject in need thereof, may result in a change, e.g., a decrease in TETRAS-mADL performance subscore score of the subject of 2 points or greater when the compound of formula (I) is administered at a dose of about 60 mg once daily after an initial titration period.

In some embodiments, methods of treating a disease or condition relating to aberrant function or activity of a T-type calcium channel, e.g., essential tremor, in a subject in need thereof, may result in a change, e.g., a decrease in mADL11 performance subscore score of the subject of 2 points or greater when the compound of formula (I) is administered at a dose of about 60 mg once daily after an initial titration period.

In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore score of the subject of at least 1 point, at least 2 points, at least 3 points, at least 4 points, at least 5 points, at least 6 points, at least 7 points, at least 8 points, at least 9 points or at least 10 points when the compound of formula (I) is administered at a dose of about 100 mg once daily after an initial titration period.

In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore score of the subject of 2 points or greater when compound of formula (I) is administered at a dose of about 100 mg once daily after an initial titration period.

In various aspects, the methods disclosed herein result in an EEG sigma frequency band reduction during NREM sleep in the subject, such as a NREM sigma band frequency reduction from a baseline of about 0.4 to 0.7, such as about 0.5 to about 0.6, or about 0.5. In certain embodiments, the methods disclosed herein result in an EEG gamma frequency band reduction during wake in an EO condition or an EC condition in the subject, such as a gamma frequency band reduction as compared to a baseline gamma frequency band of at least about 25%, such as, for example, about 50% reduction.

In certain embodiments, the methods disclosed herein result in an EEG sigma frequency reduction during NREM sleep and/or an EEG gamma frequency band reduction during an EO or an EC condition in the subject when the subject is administered a dosage of the compound of formula (I) or a pharmaceutically acceptable salt thereof resulting in a Cₘₐₓ of from about 30 ng/ml to about 470 ng/ml, such as a Cₘₐₓ ranging from about 30 to about 50 ng/ml, from about 80 to about 130 ng/mL, from about 130 to about 222 ng/ml, from about 180 to about 300 ng/mL, from about 230 to 380 ng/mL, or from about 280 to about 470 ng/ml. In certain embodiments, the methods disclosed herein result in an EEG sigma frequency reduction during NREM sleep and/or an EEG gamma frequency band reduction during an EO or an EC condition in the subject when the subject is administered a dosage of the compound of formula (I) or a pharmaceutically acceptable salt thereof resulting in an AUC₂₄ ranging from about 490 ng*h/mL to about 5800 ng*h/mL, such as an AUC₂₄ ranging from about 490 to 820 ng*h/mL, from about 1220 to 2030 ng*h/mL, from about 2000 to 3330 ng*h/mL, from about 2440 to 4070 ng*h/mL, from about 2820 to 4700 ng*h/mL, or from about 3480 to 5800 ng*h/mL.

In certain embodiments, the methods disclosed herein result in a NREM sigma frequency reduction in the subject when the subject is administered a dosage of the compound of formula (I) or a pharmaceutically acceptable salt thereof resulting in a Cₘₐₓ of about 5 ng/ml to about 470 ng/ml, such as a Cₘₐₓ of about 180 to about 300 ng/ml. In certain embodiments, the methods disclosed herein result in a NREM sigma frequency reduction in the subject when the subject is administered a dosage of the compound of formula (I) or a pharmaceutically acceptable salt thereof resulting in an average plasma concentration (Cₐᵥₑ) during the EEG recording (i.e., over a period of about 24 hours) of about 10 ng/mL to about 200 ng/ml, such as a Cₐᵥₑ of about 12 to about 150 ng/ml. In certain embodiments, the methods disclosed herein result in an EO or EC gamma frequency band reduction in the subject when the subject is administered a dosage of the compound of formula (I) or a pharmaceutically acceptable salt thereof resulting in a Cₘₐₓ of the compound of about, such as a Cₘₐₓ of about 280 to about 470 ng/mL. In certain embodiments, the methods disclosed herein result in an EO or EC gamma frequency band reduction in the subject when the subject is administered a dosage of the compound of formula (I) or a pharmaceutically acceptable salt thereof resulting in a plasma concentration of about 75 ng/ml to about 310 ng/mL, such as a plasma concentration of about 90 to about 190 ng/mL.

It certain aspects of all embodiments of titrated dosing schedules disclosed herein, it is possible to increase the maximum titrated dose, including, for example, increasing the maximum titrated dose above 120 mg in one or more additional titration steps, provided the subject is able to safely tolerate the higher dose.

In certain aspects of all of the embodiments of titrated dosing schedules disclosed herein, the maximum titrated dosage achieved is greater than 20 mg, greater than 40 mg, such as about 60 mg, about 80 mg, about 100 mg, about 120 mg, about 130 mg, about 140 mg, about 150 mg, about 160 mg, about 170 mg, about 180 mg, about 190 mg, about 200 mg, about 210 mg, or about 220 mg. In certain aspects of the disclosure, the maximum dosage for a subject is, for example, 40 mg, 60 mg, or 80 mg if the subject has achieved a desired therapeutic outcome.

In certain aspects of all of the embodiments of titrated dosing schedules disclosed herein, the maximum titrated dosage is reached in 42 days or less, such as 31 days or less, 28 days or less, 18 days or less, 10 days or less, or 7 days or less. In certain embodiments, the maximum titrated dosage is reached in about 10 to about 42 days, such as, for example, about 36-42 days, about 22-28 days, about 16-18 days, about 10-12 days, or about 7-10 days.

### Dosage Forms and Compositions

In some embodiments, the compound of formula (I) or a pharmaceutically acceptable salt thereof, e.g., the compound of formula (II) may be administered as a part of a dosage form or in a pharmaceutical composition.

In some embodiments, a composition that can be used in a method described herein may be a pharmaceutical composition comprising the compound of formula (I) or a pharmaceutically acceptable salt thereof, and an excipient that functions to modify the release rate of the compound of formula (I) or a pharmaceutically acceptable salt thereof. In some embodiments, the pharmaceutical composition may be a swellable core technology formulation.

In certain embodiments, a dosage form that can be used in a method described herein may be an oral dosage form comprising: the compound of formula (I) or a pharmaceutically acceptable salt thereof (e.g., the compound of formula (II)); and a modified-release polymer (e.g., a controlled-release polymer, hydrophilic matrix polymers, e.g., an HPMC polymer, hydrophobic matrix polymers (e.g., ethyl cellulose, ethocel), or polyacrylate polymers (e.g., Eudragit RL100, Eudragit R$100)).

In other embodiments, a dosage form that can be used in a method described herein may be a dosage form or composition comprising the compound of formula (I) or a pharmaceutically acceptable salt thereof (e.g., the compound of formula (II)) and a modified-release polymer (e.g., a controlled-release polymer, hydrophilic matrix polymers, e.g., an HPMC polymer, hydrophobic matrix polymers (e.g., ethyl cellulose, ethocel), or polyacrylate polymers (e.g., Eudragit RL100, Eudragit RS100)), for example, in an amount sufficient to modify the release rate of the compound of formula (I) or a pharmaceutically acceptable salt thereof (e.g., the compound of formula (II)) upon administration to the subject.

In some embodiments, the dosage form comprises from about 0.9% by weight to about 40% by weight (e.g., from about 0.9% by weight to about 30%, from about 1% by weight to about 25% by weight, from about 2% by weight to about 25% by weight, from about 3% by weight to about 20% by weight, from about 4% by weight to about 20% by weight, from about 5% by weight to about 20% by weight, from about 5% by weight to about 15% by weight, from about 5% by weight to about 10% by weight, or about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 40% by weight) of the compound of formula (I) or a pharmaceutically acceptable salt thereof (e.g., the compound of formula (II)). In some embodiments, the dosage form comprises about 30% by weight to about 40% by weight of the compound of formula (I) or a pharmaceutically acceptable salt thereof (e.g., the compound of formula (II)).

In some embodiments, the dosage form comprises from about 14% by weight to about 25% by weight of the compound of formula (I) or a pharmaceutically acceptable salt thereof (e.g., the compound of formula (II)). In some embodiments, the dosage form comprises from about 19% by weight to about 20% by weight of the compound of formula (I) or a pharmaceutically acceptable salt thereof (e.g., the compound of formula (II)). In some embodiments, the dosage form comprises from about 21% by weight to about 22% by weight of the compound of formula (I) or a pharmaceutically acceptable salt thereof (e.g., the compound of formula (II)). In some embodiments, the dosage form comprises from about 4% by weight to about 15% by weight of the compound of formula (I) or a pharmaceutically acceptable salt thereof (e.g., the compound of formula (II)). In some embodiments, the dosage form comprises from about 4% by weight to about 10% by weight of the compound of formula (I) or a pharmaceutically acceptable salt thereof (e.g., the compound of formula (II)). In some embodiments, the dosage form comprises form about 4% by weight to about 5% by weight of the compound of formula (I) or a pharmaceutically acceptable salt thereof (e.g., the compound of formula (II)). In some embodiments, the dosage form comprises from about 5% by weight to about 6% by weight of the compound of formula (I) or a pharmaceutically acceptable salt thereof (e.g., the compound of formula (II)). In some embodiments, the dosage form comprises from about 9% by weight to about 10% by weight of the compound of formula (I) or a pharmaceutically acceptable salt thereof (e.g., the compound of formula (II)).

In other embodiments, a dosage form that can be used in a method described herein may be a dosage form or composition comprising from about 1 mg to about 120 mg (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg) of the compound of formula (I) or a pharmaceutically acceptable salt thereof (e.g., the compound of formula (II)) and a modified-release polymer (e.g., a controlled-release polymer, hydrophilic matrix polymers, e.g., an HPMC polymer, hydrophobic matrix polymers (e.g., ethyl cellulose, ethocel), or polyacrylate polymers (e.g., Eudragit RL100, Eudragit RS100)), for example, in an amount sufficient to modify the release rate of the compound of formula (I) or a pharmaceutically acceptable salt thereof (e.g., the compound of formula (II)) upon administration to the subject.

In other embodiments, the dosage form comprises from about 4 mg to about 6 mg (e.g., about 5 mg) of the compound of formula (I) or a pharmaceutically acceptable salt thereof (e.g., the compound of formula (II)). In certain embodiments, the dosage form comprises from about 15 mg to about 25 mg (e.g., about 20 mg) of the compound of formula (I) or a pharmaceutically acceptable salt thereof (e.g., the compound of formula (II)). In some embodiments, the dosage form comprises from about 5 mg to about 15 mg (e.g., about 10 mg) of the compound of formula (I) or a pharmaceutically acceptable salt thereof (e.g., the compound of formula (II)). In other embodiments, the dosage form comprises from about 25 mg to about 35 mg (e.g., about 30 mg) of the compound of formula (I) or a pharmaceutically acceptable salt thereof (e.g., the compound of formula (II)). In certain embodiments, the dosage form comprises from about 35 mg to about 45 mg (e.g., about 40 mg) of the compound of formula (I) or a pharmaceutically acceptable salt thereof (e.g., the compound of formula (II)). In certain embodiments, the dosage form comprises from about 45 mg to about 55 mg (e.g., about 50 mg) of the compound of formula (I) or a pharmaceutically acceptable salt thereof (e.g., the compound of formula (II)). In some embodiments, the dosage form comprises from about 55 mg to about 65 mg (e.g., about 60 mg) of the compound of formula (I) or a pharmaceutically acceptable salt thereof (e.g., the compound of formula (II)). In other embodiments, the dosage form comprises from about 65 mg to about 75 mg (e.g., about 70 mg) of the compound of formula (I) or a pharmaceutically acceptable salt thereof (e.g., the compound of formula (II)). In certain embodiments, the dosage form comprises from about 75 mg to about 85 mg (e.g., about 80 mg) of the compound of formula (I) or a pharmaceutically acceptable salt thereof (e.g., the compound of formula (II)). In certain embodiments, the dosage form comprises from about 85 mg to about 95 mg (e.g., about 90 mg) of the compound of formula (I) or a pharmaceutically acceptable salt thereof (e.g., the compound of formula (II)). In some embodiments, the dosage form comprises from about 95 mg to about 105 mg (e.g., about 100 mg) of the compound of formula (I) or a pharmaceutically acceptable salt thereof (e.g., the compound of formula (II)). In other embodiments, the dosage form comprises from about 105 mg to about 115 mg (e.g., about 110 mg) of the compound of formula (I) or a pharmaceutically acceptable salt thereof (e.g., the compound of formula (II)). In certain embodiments, the dosage form comprises from about 115 mg to about 125 mg (e.g., about 120 mg) of the compound of formula (I) or a pharmaceutically acceptable salt thereof (e.g., the compound of formula (II)).

In some embodiments, the dosage form comprises from about 55 mg to 65 mg of a modified-release polymer (e.g., an HPMC polymer). In some embodiments, the dosage form comprises from about 10% by weight to about 70% by weight of the modified-release polymer (e.g., an HPMC polymer). In some embodiments, the dosage form comprises from about 50% by weight to about 60% by weight of the modified-release polymer (e.g., an HPMC polymer).

In some embodiments, the dosage form further comprises a diluent. In some embodiments, the diluent comprises microcrystalline cellulose. In some embodiments, the dosage form comprises from about 15 mg to 40 mg (e.g., from about 15 mg to about 25 mg, from about 20 mg to about 25 mg, from about 25 mg to about 30 mg, from about 30 mg to about 40 mg) microcrystalline cellulose. In some embodiments, the dosage form comprises from about 15 mg to about 25 mg microcrystalline cellulose. In some embodiments, the dosage form comprises from about 30 mg to about 40 mg microcrystalline cellulose. In some embodiments, the dosage form comprises from about 15% to about 35% by weight (e.g., from about 15% to about 20%, from about 20% to about 25%, from about 25% to about 30%, from about 30% to about 35% by weight) microcrystalline cellulose.

In some embodiments, the dosage form further comprises a glidant. In some embodiments, the glidant comprises colloidal silicon dioxide. In some embodiments, the dosage form further comprises a lubricant. In some embodiments, the lubricant comprises magnesium stearate. In some embodiments, the dosage form further comprises a coating.

In some embodiments, about 80% of the compound of formula (I) or a pharmaceutically acceptable salt is released within 7 hours upon administration to a subject. In certain embodiments, about 80% of the compound of formula (I) or a pharmaceutically acceptable salt thereof is released in 7 hours using USP apparatus type-I, media containing 900 mL 0.1 M HCl, and a paddle speed of 100 rpm.

In some embodiments, the dosage form, upon administration to a subject, has a lower Cₘₐₓ value than a reference oral dosage form (e.g., a dosage form with any intended release rate profile e.g., modified release rate profile, a dosage form that does not have a modified release rate profile, e.g., an HPMC polymer). In some embodiments, the dosage form, upon administration to a subject, has a greater tₘₐₓ value than a reference oral dosage form (e.g., a dosage form with any intended release rate profile e.g., modified release rate profile, a dosage form that does not have a modified release rate profile, e.g., an HPMC polymer).

In some embodiments, the dosage form is administered to a patient once daily. In certain embodiments, the dosage form is administered to a patient twice daily. In some embodiments, the dosage form is a tablet. In other embodiments, the dosage form is a capsule. In certain embodiments, the dosage form is a suspension.

In some embodiments, a dosage form that can be used in a method described herein may be an oral dosage form (e.g., particulate) comprising: from about 15 mg to 25 mg of the compound of formula (I) or a pharmaceutically acceptable salt thereof (e.g., the compound of formula (II)); and from about 55 mg to 65 mg of an HPMC polymer.

In other embodiments, a dosage form that can be used in a method described herein may be an oral dosage form (e.g., particulate) comprising from about 14% by weight to about 25% by weight of the compound of formula (I) or a pharmaceutically acceptable salt thereof (e.g., the compound of formula (II)); and from about 53% to about 64% by weight of an HPMC polymer.

In certain embodiments, a dosage form that can be used in a method described herein may be an oral dosage form (e.g., particulate) comprising from about 3 mg to 8 mg of the compound of formula (I) or a pharmaceutically acceptable salt thereof (e.g., the compound of formula (II)); and from about 55 mg to 65 mg of an HPMC polymer.

In some embodiments, a dosage form that can be used in a method described herein may be an oral dosage form (e.g., particulate) comprising from about 3% by weight to about 8% by weight of the compound of formula (I) or a pharmaceutically acceptable salt thereof (e.g., the compound of formula (II)); and from about 53% to about 64% by weight of an HPMC polymer.

In other embodiments, a dosage form that can be used in a method described herein may be an oral (e.g., particulate) composition comprising the compound of formula (I) or a pharmaceutically acceptable salt thereof (e.g., the compound of formula (II)); and a modified-release polymer (e.g., a controlled-release polymer, e.g., an HPMC polymer as a hydrophilic matrix polymer).

In some embodiments, the composition comprises from about 0.9% by weight to about 40% by weight of the compound of formula (I) or a pharmaceutically acceptable salt thereof (e.g., the compound of formula (II)). In some embodiments, the composition comprises from about 14% by weight to about 25% by weight of the compound of formula (I) or a pharmaceutically acceptable salt thereof (e.g., the compound of formula (II)). In some embodiments, the composition comprises about 19% by weight to about 20% by weight of the compound of formula (I) or a pharmaceutically acceptable salt thereof (e.g., the compound of formula (II)). In some embodiments, the composition comprises about 21% by weight to about 22% by weight of the compound of formula (I) or a pharmaceutically acceptable salt thereof (e.g., the compound of formula (II)). In some embodiments, the composition comprises from about 4% by weight to about 15% by weight of the compound of formula (I) or a pharmaceutically acceptable salt thereof (e.g., the compound of formula (II)). In some embodiments, the composition comprises from about 4% by weight to about 10% by weight of the compound of formula (I) or a pharmaceutically acceptable salt thereof (e.g., the compound of formula (II)). In some embodiments, the composition comprises about 4% by weight to about 5% by weight of the compound of formula (I) or a pharmaceutically acceptable salt thereof (e.g., the compound of formula (II)). In some embodiments, the composition comprises about 5% by weight to about 6% by weight of the compound of formula (I) or a pharmaceutically acceptable salt thereof (e.g., the compound of formula (II)). In some embodiments, the composition comprises about 9% by weight to about 10% by weight of the compound of formula (I) or a pharmaceutically acceptable salt thereof (e.g., the compound of formula (II)).

In some embodiments, the composition comprises from about 1 mg to about 120 mg (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg) of the compound of formula (I) or a pharmaceutically acceptable salt thereof (e.g., the compound of formula (II)). In certain embodiments, the composition comprises from about 4 mg to about 6 mg (e.g., about 5 mg) of the compound of formula (I) or a pharmaceutically acceptable salt thereof (e.g., the compound of formula (II)). In other embodiments, the composition comprises from about 15 mg to about 25 mg (e.g., about 20 mg) of the compound of formula (I) or a pharmaceutically acceptable salt thereof (e.g., the compound of formula (II)). In certain embodiments, the composition comprises from about 25 mg to about 35 mg (e.g., about 30 mg) of the compound of formula (I) or a pharmaceutically acceptable salt thereof (e.g., the compound of formula (II)). In other embodiments, the composition comprises from about 35 mg to about 45 mg (e.g., about 40 mg) of the compound of formula (I) or a pharmaceutically acceptable salt thereof (e.g., the compound of formula (II)). In certain embodiments, the composition comprises from about 45 mg to about 55 mg (e.g., about 50 mg) of the compound of formula (I) or a pharmaceutically acceptable salt thereof (e.g., the compound of formula (II)). In some embodiments, the composition comprises from about 55 mg to about 65 mg (e.g., about 60 mg) of the compound of formula (I) or a pharmaceutically acceptable salt thereof (e.g., the compound of formula (II)). In other embodiments, the composition comprises from about 65 mg to about 75 mg (e.g., about 70 mg) of the compound of formula (I) or a pharmaceutically acceptable salt thereof (e.g., the compound of formula (II)). In certain embodiments, the composition comprises from about 75 mg to about 85 mg (e.g., about 80 mg) of the compound of formula (I) or a pharmaceutically acceptable salt thereof (e.g., the compound of formula (II)). In certain embodiments, the composition comprises from about 85 mg to about 95 mg (e.g., about 90 mg) of the compound of formula (I) or a pharmaceutically acceptable salt thereof (e.g., the compound of formula (II)). In some embodiments, the composition comprises from about 95 mg to about 105 mg (e.g., about 100 mg) of the compound of formula (I) or a pharmaceutically acceptable salt thereof (e.g., the compound of formula (II)). In other embodiments, the composition comprises from about 105 mg to about 115 mg (e.g., about 110 mg) of the compound of formula (I) or a pharmaceutically acceptable salt thereof (e.g., the compound of formula (II)). In certain embodiments, the composition comprises from about 115 mg to about 125 mg (e.g., about 120 mg) of the compound of formula (I) or a pharmaceutically acceptable salt thereof (e.g., the compound of formula (II)).

In some embodiments, the composition comprises a diluent. In some embodiments, the diluent comprises microcrystalline cellulose. In other embodiments, the composition comprises from about 15 mg to 40 mg (e.g., from about 15 mg to about 25 mg, from about 20 mg to about 25 mg, from about 25 mg to about 30 mg, from about 30 mg to about 40 mg), microcrystalline cellulose. In some embodiments, the composition comprises from about 15% to about 35% by weight (e.g., from about 15% to about 20%, from about 20% to about 25%, from 25% to about 30%, from 30% to about 35% by weight) microcrystalline cellulose.

In some embodiments, the composition comprises from about 15 mg to about 25 mg microcrystalline cellulose. In some embodiments, the composition comprises from about 30 mg to about 40 mg microcrystalline cellulose. In some embodiments, the composition further comprises a glidant. In some embodiments, the glidant comprises colloidal silicon dioxide. In some embodiments, the composition further comprises a lubricant. In some embodiments, the lubricant comprises magnesium stearate. In some embodiments, the composition further comprises a coating. In some embodiments, the compound of formula (I) or (II), including, for example, the compound of Form C or Form B, is stable within the formulation at about 25° C. at 60% relative humidity for at least 24 months. In some embodiments, the compound is stable at about 25° C. at 60% relative humidity for at least 36 months. In some embodiments, the compound is stable at about 25° C. at 60% relative humidity for at least 48 months. In other embodiments, the compound is stable at about 25° C. at 60% relative humidity for at least 60 months. In some embodiments, the compound is stable at about 40° C. at 75% relative humidity for at least 6 months.

In other embodiments of the oral dosage forms or compositions described herein, the compound of formula (I) or a pharmaceutically acceptable salt thereof (e.g., the compound of formula (II)) is a crystalline form. In certain embodiments, the crystalline form is a crystalline form as described herein, such as crystalline Form C or crystalline Form B. In other embodiments, the crystalline form is crystalline Form D. In certain embodiments, the crystalline form is a crystalline form previously or contemporaneously described, such as crystalline Pattern B, crystalline Pattern C or crystalline Pattern D. Crystalline Pattern B and crystalline Pattern C are described, e.g., in WO 2021/007487, the entire contents of which are hereby incorporated herein by reference. Crystalline Pattern D is characterized by an x-ray powder diffraction (XRPD) pattern comprising at least one peak at the diffraction angle (°2θ) selected from the group consisting of a peak at approximately 12.0°; a peak at approximately 15.6°; a peak at approximately 16.7°; a peak at approximately 19.8°; a peak at approximately 21.2°; a peak at approximately 24.1°; a peak at approximately 25.2°; a peak at approximately 27.3°; and a peak at approximately 30.2°.

### Crystalline Form of the Compound of Formula (II)

A crystalline form of the compound of formula (II) used in the method as described herein may exhibit an X-ray powder diffraction (XRPD) pattern comprising at least one peak selected from peaks at the following diffraction angles (2θ): 26.6±0.2, 16.2±0.2, 17.4±0.2, 22.6±0.2, 11.5±0.2, 23.9±0.2, 18.3±0.2, 19.2±0.2, 18.5±0.2 or 20.0±0.2. In certain embodiments, a crystalline form of the compound of formula (II) disclosed herein may exhibit an XRPD pattern comprising peaks at the following diffraction angles (2θ): 16.2±0.2, 17.4±0.2, and 26.6±0.2.

In some embodiments, the crystalline form exhibits an XRPD pattern comprising peaks at the following diffraction angles (2θ): 11.5±0.2, 16.2±0.2, 17.4±0.2, 22.6±0.2, and 26.6±0.2. In other embodiments, the crystalline form exhibits an XRPD pattern comprising peaks at the following diffraction angles (2θ): 11.5±0.2, 16.2±0.2, 17.4±0.2, 18.3±0.2, 18.5±0.2, 19.2±0.2, 20.0±0.2, 22.6±0.2, 23.9±0.2, and 26.6±0.2. In some embodiments, the XRPD pattern was obtained using Cu Kα radiation. In certain embodiments, the crystalline form has a melting point onset as determined by differential scanning calorimetry at about 226.6° C.

A crystalline form of the compound of formula (II) used in the method as described herein may exhibit an XRPD pattern comprising at least one peak selected from peaks at the following diffraction angles (2θ): 21.9±0.2, 18.5±0.2, 17.8±0.2, 10.2±0.2, 20.5±0.2, 25.2±0.2, 16.9±0.2, 24.2±0.2, 28.6±0.2 or 21.2±0.2. In certain embodiments, a crystalline form of the compound of formula (II) disclosed herein may exhibit an XRPD pattern comprising peaks at the following diffraction angles (2θ): 21.9±0.2, 18.5±0.2, and 17.8±0.2.

In some embodiments, the crystalline form exhibits an XRPD pattern comprising peaks at the following diffraction angles (2θ): 21.9±0.2, 18.5±0.2, 17.8±0.2, 10.2±0.2, and 20.5±0.2. In other embodiments, the crystalline form exhibits an XRPD pattern comprising peaks at the following diffraction angles (2θ): 21.9±0.2, 18.5±0.2, 17.8±0.2, 10.2±0.2, 20.5±0.2, 25.2±0.2, 16.9±0.2, 24.2±0.2, 28.6±0.2, and 21.2±0.2. In some embodiments, the powder XRPD was obtained using Cu Kα radiation. In certain embodiments, the crystalline form has a melting point onset as determined by differential scanning calorimetry at about 97.9° C., 131.6° C., 223.7° C., 83.8° C., 128.9° C., 168.9° C., or 224.4° C.

### Immediate-Release Formulations

In some embodiments, a dosage form or composition that can be used in a method described herein may be a dosage form or composition comprising the compound of formula (I) or a pharmaceutically acceptable salt thereof (e.g., the compound of formula (II)), where the compound of formula (I) or a pharmaceutically acceptable salt thereof (e.g., the compound of formula (II)) is released immediately upon an administration to the subject.

In other embodiments, a dosage form that can be used in a method described herein may be an oral capsule for immediate release comprising from about 15 mg to about 20 mg of the compound of formula (I) or a pharmaceutically acceptable salt thereof (e.g., the compound of formula (II)); from about 75 mg to 85 mg diluent; from about 2 mg to 10 mg binder; from about 1% to about 5% disintegrant; and from about 0.1 mg to 5 mg lubricant.

### Administrations

In some embodiments, the dosage form is administered to the subject more than once a day (e.g., twice a day, three times a day, or four times a day).

In some embodiments, the dosage form is administered to the subject once a day (e.g., one 20 mg tablet once a day, two 20 mg tablets (or one 40 mg tablet) once a day, or three 20 mg tablets (or one 60 mg tablet) once a day). In some embodiments, the dosage form is administered to the subject twice a day (e.g., one 10 mg tablet twice a day, one 20 mg tablet twice a day, two 20 mg tablets twice a day, three 20 mg tablets twice a day). In some embodiments, the dosage form is administered to the subject every other day, twice a week, or once a week. In certain embodiments, a dose of about 1 mg to 60 mg, such as 20 mg to 40 mg, of the compound of formula (I) or a pharmaceutically acceptable salt thereof (e.g., the compound of formula (II)) is administered to the subject daily. In other embodiments, a dose of about 15 mg to 25 mg of the compound of formula (I) or a pharmaceutically acceptable salt thereof (e.g., the compound of formula (II)) is administered to the subject daily. In certain embodiments, a dose of about 30 mg to 40 mg of the compound of formula (I) or a pharmaceutically acceptable salt thereof (e.g., the compound of formula (II)) is administered to the subject daily.

In some embodiments, the dosage form, upon administration to the subject, has a reduced Cₘₐₓ value in comparison to a reference oral dosage form (e.g., a dosage form with any intended release rate profile e.g., modified release rate profile, a dosage form that does not have a modified release rate profile, e.g., an HPMC polymer). In some embodiments, the dosage form, upon administration to the subject, has a greater tₘₐₓ value than a reference oral dosage form (e.g., a dosage form with any intended release rate profile e.g., modified release rate profile, a dosage form that does not have a modified release rate profile, e.g., an HPMC polymer).

### Tremor

The methods described herein can be used to treat tremor, for example a dosage or composition disclosed herein can be used to treat cerebellar tremor or intention tremor, dystonic tremor, essential tremor, orthostatic tremor, parkinsonian tremor, physiological tremor, or rubral tremor. Tremor includes hereditary, degenerative, and idiopathic disorders such as Wilson's disease (hereditary), Parkinson's disease (degenerative), and essential tremor (idiopathic); metabolic diseases; peripheral neuropathies (associated with Charcot-Marie-Tooth, Roussy-Levy, diabetes mellitus, complex regional pain syndrome); toxins (nicotine, mercury, lead, carbon monoxide, manganese, arsenic, toluene); drug-induced (neuroleptics tricyclics, lithium, cocaine, alcohol, adrenaline, bronchodilators, theophylline, caffeine, steroids, valproate, amiodarone, thyroid hormones, vincristine); and psychogenic disorders. Clinical tremor can be a neuropathic tremor, and can be classified into physiologic tremor, enhanced physiologic tremor, essential tremor syndromes (including classical essential tremor), primary orthostatic tremor, task-and position-specific tremor, dystonic tremor, parkinsonian tremor, cerebellar tremor, Holmes' tremor (i.e., rubral tremor), palatal tremor, toxic or drug-induced tremor, and psychogenic tremor. The tremor may be familial tremor.

Tremor is an involuntary, rhythmic, oscillation of one or more body parts (e.g., hands, arms, eyes, face, head, vocal folds, trunk, and/or legs).

Cerebellar tremor or intention tremor is a slow, broad tremor of the extremities that occurs after a purposeful movement. Cerebellar tremor is caused by lesions in or damage to the cerebellum or pathways resulting from, e.g., tumor, stroke or other focal lesion disease (e.g., multiple sclerosis) or a neurodegenerative disease.

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occur irregularly and often can be relieved by complete rest or certain sensory maneuvers.

Essential tremor or benign essential tremor is the most common type of tremor. Essential tremor may be mild and nonprogressive in some, or may be slowly progressive, starting on one side of the body but typically affecting both sides. Essential tremor may also be moderate or severe. The hands are most often affected, but the head, voice, tongue, legs, and trunk may also be involved. Tremor frequency may decrease as a person ages, but, alternatively, severity may increase. Heightened emotion, stress, fever, other illness, physical exhaustion, or low blood sugar may trigger tremors and/or increase their severity. Symptoms generally evolve over time and can be both visible and persistent following onset.

Orthostatic tremor is characterized by fast (e.g., greater than 12 Hz) rhythmic muscle contractions that occurs in the legs and trunk immediately after standing. Cramps are felt in the thighs and legs and the patient may shake uncontrollably when asked to stand in one spot. Orthostatic tremor may occur in patients with essential tremor.

Parkinsonian tremor is caused by damage to structures within the brain that control movement. Parkinsonian tremor is typically seen as a “pill-rolling” action of the hands that may also affect the chin, lips, legs, and trunk. Onset of parkinsonian tremor typically begins after age 60. Movement starts in one limb or on one side of the body and can progress to include the other side.

Rubral tremor is characterized by coarse slow tremor which can be present at rest, at posture, and with intention. The tremor is associated with conditions that affect the red nucleus in the midbrain, such as a stroke.

In some embodiments, the tremor is selected from essential tremor, Parkinson's tremor, or Cerebellar tremor. In an embodiment, the tremor is essential tremor. In various embodiments, the essential tremor is with or without intention tremor. In an embodiment, the essential tremor is without intention tremor.

The efficacy of the compound or composition described herein for treating essential tremor can be measured by methods known in the art, such as the methods described in the following references: Ferreira, J. J. et al., “MDS Evidence-Based Review of Treatments for Essential Tremor,” Mov. Disord. 2019 July; 34(7):950-958; Elble, R. et al., “Task Force Report: Scales for Screening and Evaluating Tremor,” Mov. Disord. 2013 November; 28(13):1793-800; Deuschl G. et al., “Treatment of patients with essential tremor,” Lancet Neurol. 2011; 10:148-61; and Reich S. G. et al., “Essential Tremor,” Med. Clin. N. Am. 2019; 103:351-356. The disclosures of the references are herein incorporated in their entirety.

In some embodiments, the methods described herein result in at least 25% reduction in the upper limb tremor score, wherein the tremor score may be converted to amplitude, as compared to a baseline. For example, in certain embodiments, the methods described herein result in about 40% mean reduction in tremor amplitude as measured by The Essential Tremor Rating Assessment Scale (TETRAS) upper limb score, described, for example, in Elble, R. J., “The Essential Tremor Rating Assessment Scale,” J. Neurol. Neuromed. 2016; 1(4):34-38. In some embodiments, the methods described herein result in at least 25% reduction in TETRAS performance score as compared to the baseline. In some embodiments, the methods described herein result in at least 35% average reduction in symptom severity as compared to the baseline, as measured by TETRAS performance score.

### Combination Therapy

In the context of the present disclosure, the compound of formula (I), or a pharmaceutically acceptable salt thereof, may be administered in combination with one or more other agent or therapy. For example, the compound of formula (I), or a pharmaceutically acceptable salt thereof, may be administered to a subject in combination with one or more tremor medications. Exemplary tremor medications which may be administered in combination with compound of formula (I), or a pharmaceutically acceptable salt thereof, in the context of the present disclosure include propranolol, primidone, clonazepam, diazepam, lorazepam, alprazolam, gabapentin, topiramate, topamax, neurontin, atenolol, klonopin, alprazolam, nebivolol, carbidopa/levodopa, clonazepam, hydrochlorothiazide/metoprolol, gabapentin enacarbil, labetalol, lactulose, lamotrigine, metoprolol, nadolol, hydrochlorothiazide, and zonisamide. In one embodiment, the tremor medication is propranolol. In another embodiment, the tremor medication is primidone.

In an embodiment, tremor medications include propranolol, primidone, clonazepam, diazepam, lorazepam, alprazolam, gabapentin, topiramate, topamax, neurontin, atenolol, klonopin, alprazolam, nebivolol, carbidopa/levodopa, clonazepam, hydrochlorothiazide/metoprolol, gabapentin enacarbil, labetalol, lactulose, lamotrigine, metoprolol, nadolol, hydrochlorothiazide, and zonisamide.

### EXAMPLES

In order that the embodiments described herein may be more fully understood, the following examples are set forth. The synthetic and biological examples described in this application are offered to illustrate the compounds, pharmaceutical compositions, and methods provided herein and are not to be construed in any way as limiting their scope.

### Example 1: Phase 2 Trial to Evaluate the Efficacy of the Compound of Formula (I) in the Treatment of Adults with Essential Tremor

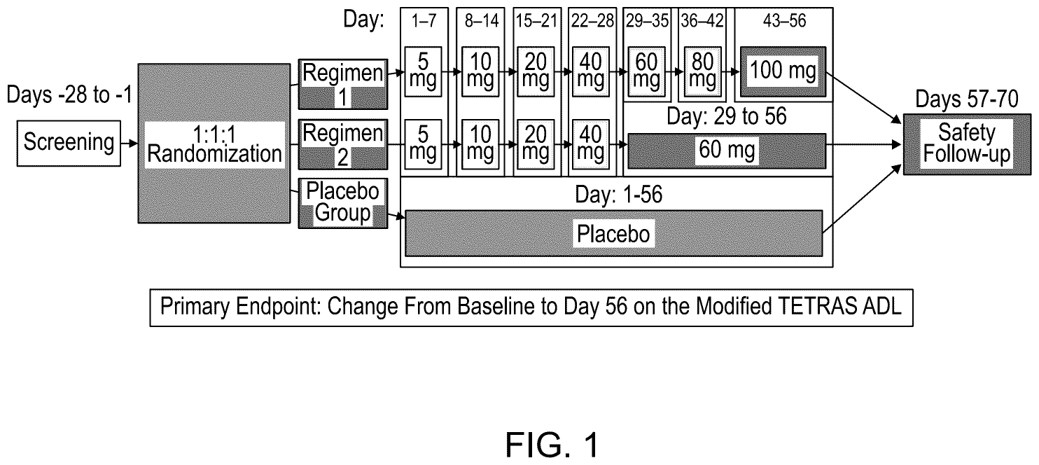

This trial is a Phase 2 trial to evaluate the efficacy and safety/tolerability of titration of Compound 1 to 60 mg or 100 mg compared to placebo in participants with moderate to severe ET. Severity of ET in this study was defined by eligibility criteria on TETRAS-ADL, CGI-S, and TETRAS-PS items 6 and 7. This dose range finding trial is comprised of three parts. The first part was randomized, double-blinded (DB), and placebo-controlled, the second part was an optional extension that consisted of an Extension DB Lead-in Period followed by an Extension Open-label (OL) Period. Further, there was also a third part available for completers of the Day 99 visit, which third part was comprised of a blinded cross-over period (referred to as cross-over period) after re-randomization for 6 weeks, after which all completers would receive formula (I) in an open-label fashion. In part one of the trial, eligible participants were randomized to 1 of 2 dose levels of Compound 1 (60 mg or 100 mg) or to placebo. The primary objective of this trial was to evaluate the efficacy of Compound 1 compared to placebo after 56 days (end of randomized, DB, placebo-controlled part) of dosing in participants with ET with moderate to severe tremor utilizing modified ADL as the primary efficacy endpoint. Secondary and exploratory objectives include further efficacy evaluations, an evaluation of the safety and tolerability, and determination of plasma concentrations of formula (I) and its metabolites. The design of the Phase 2 trial is illustrated in FIG. 1.

| | |
|---|---|
| Objective | Endpoint |

Primary

| | |
|---|---|
| To evaluate the efficacy of | Change from baseline to Day 56 on |
| Compound 1 compared to placebo | the modified ADL |
| in participants with ET | |

Secondary
Efficacy

| | |
|---|---|
| To further evaluate the efficacy | Change from baseline to Day 56 on |
| of Compound 1 compared to | the Clinical Global Impression- |
| placebo in participants with | Severity (CGI-S) |
| ET | Clinical Global Impression- |
| | Improvement (CGI-I) score at Day |
| | 56 |
| | Change from baseline to Day 56 on |
| | the following: |
| | TETRAS-ADL score |
| | TETRAS-performance |
| | Subscale (PS) total score |
| | TETRAS-upper limb (UL) |
| | score (TETRAS-PS item 4) |
| | TETRAS-combined upper |
| | limb (CUL) score (TETRAS- |
| | PS sum of items 4, 6, 7, and |
| | 8) |
| | Patient Global Impression-Change |
| | (PGI-C) score at Day 56 |
| | Change from baseline to Days 14, |
| | 28, and 42 on the following: |
| | modified ADL |
| | CGI-S |
| | TETRAS-ADL total score |
| | TETRAS-UL score |
| | TETRAS-CUL score |
| | CGI-I and PGI-C scores at Days |
| | 14, 28, and 42 |
| | Change from baseline to Day 42 on |
| | the TETRAS-PS total score |

Safety

| | |
|---|---|
| To evaluate the safety and | Incidence and severity of AEs, |
| tolerability of Compound 1 (in | including discontinuation of study |
| both the DB and the Extension | drug due to AEs |
| parts of the trial) compared to | Changes in vital sign measurements |
| placebo (in the DB part of the | Changes in clinical laboratory results |
| trial) in participants with ET | Changes in electrocardiogram (ECG) |
| | parameters |
| | Incidence of Columbia-Suicide |
| | Severity Rating Scale (C-SSRS) |
| | measured suicidal ideation or |
| | behavior |

Exploratory
Efficacy

| | |
|---|---|
| To evaluate the efficacy of | Change from baseline to Days 70, 84, |
| Compound 1 in participants with | 99, 129, 159, 189, 279, 369, and 459 |
| ET in the Extension part of the | in the following: |
| trial | modified ADL |
| | CGI-S |
| | TETRAS-ADL total score |
| | TETRAS-PS total score |
| | TETRAS-UL score |
| | TETRAS-CUL score |
| | CGI-I and PGI-C scores at Days 70, |
| | 84, 99, 129, 159, 189, 279, 369, and |
| | 459 |
| To evaluate the efficacy of | Change from re-randomization visit |
| Compound in participants with | to 7, 14, 21, 28, 35 and 42 days post |
| ET in the cross-over part of | re-randomization in: |
| the trial | TETRAS ADL response |
| | status |
| | TETRAS ADL |

PK

| | |
|---|---|
| To determine plasma | Plasma Compound 1 and metabolite |
| concentrations of Compound 1 | concentrations over time |
| and its metabolites | |
| in participants with ET | |

Participants who continued to meet all clinical trial entry criteria on Day 1 were randomized to receive DB treatment with Compound 1 or placebo every morning (QAM) from Day 1 through Day 56. Participants were randomized to 1 of 2 fixed-dose regimens or placebo in a 1:1:1 ratio. No dosage adjustments were allowed.

TABLE 1
Dosing Sequence by Regimen for Double-blind Part of the Trial

| | Dosing Sequence - Double-blind Part of the Trial | | | | | | |
|---|---|---|---|---|---|---|---|
| | Days | Days | Days | Days | Days | Days | |
| | on | on | on | on | on | on | |
| | 5 mg | 10 mg | 20 mg | 40 mg | 60 mg | 80 mg | Days on 100 mg |
| Regimen | Dose | Dose | Dose | Dose | Dose | Dose | Dose |
| Regimen 1 | 7 | 7 | 7 | 7 | 7 | 7 | 14 |
| (active drug) | | | | | | | |
| Regimen 2 | 7 | 7 | 7 | 7 | 28 | — | — |
| (active drug) | | | | | | | |

| | |
|---|---|
| Placebo | Dosed with placebo every day during Intervention Period (56 days) |

TABLE 2
Dosing Sequence by Regimen for Double-blind
Lead-in Period of Extension Part of the Trial

| | Dosing Sequence - DB Lead-in to Extension Part of the Trial | | | | | | |
|---|---|---|---|---|---|---|---|
| | | Days | | | Days | Days | Days |
| Treatment Assignment | Days on | on | Days on | Days on | on | on | on |
| in Double-blind Part | 5 mg | 10 mg | 20 mg | 40 mg | 60 mg | 80 mg | 100 mg |
| of the Trial | Dose | Dose | Dose | Dose | Dose | Dose | Dose |
| Regimen 1 | — | — | — | — | — | — | 43 |
| Compound 1 100 mg | | | | | | | |
| Regimen 2 | — | — | — | — | — | 7 | 36 |
| Compound 1 60 mg | | | | | | | |
| Placebo | 7 | 7 | 7 | 7 | 7 | 7 | 1 |

DB = double-blind; OL = open-label.

TABLE 3
Dosing Sequence by Regimen for Cross-over Period

| | Dosing Sequence - Cross-over period | | | | | |
|---|---|---|---|---|---|---|
| | Period 1 (3 weeks) | | | Period 2 (3 weeks) | | |
| | Days on | Days on | Days on | Days on | Days on | Days on |
| | 20 mg | 40 mg | 60 mg | 20 mg | 40 mg | 60 mg |
| Regimen | Dose | Dose | Dose | Dose | Dose | Dose |
| Active | Maintain dose from | | | Placebo¹ | | |
| drug | OL extension prior | | | | | |
| | to re-randomization | | | | | |
| Assigned to | Placebo¹ | | | 3 | 4 | 14 |
| Placebo | | | | | | |

¹Participants will be assessed every 7 days. If a participant receiving placebo meets the criteria to re-start drug, the participant will be re-started on drug with a dosing regimen of 20 mg dose for 3 days, 40 mg dose for 4 days and 60 mg dose for the remainder of the period. If a participant receiving placebo in Period 1 restarts active drug, the dose will be 60 mg dose for the 3 weeks in Period 2. In Period 2, if participant receiving placebo does not re-start drug before the end of the period, they will dose titrate in the open label extension.

Throughout the Intervention Period (Day 1 to Day 56), participants who chose to stop dosing or were required to stop dosing entered the Safety Follow-up Period. All participants were asked to return to the clinic for the Safety Follow-up Visit 14 days after their last dose of study drug except for those that chose to enroll in the optional Extension part of the trial (see below).

On Day 1, participants received 6 tablets of study drug QAM, and TETRAS-PS, TETRAS-ADL, and CGI-S were performed before dosing. Additional assessments performed pre-dose included pharmacokinetic (PK) sampling.

Participants continued taking 6 tablets of study drug QAM throughout the DB part of the trial.

On Days 14, 28, 42, and 56, participants returned to the clinic for PK sampling, dosing, efficacy, and safety assessments. Key efficacy assessments at these visits included TETRAS-PS, TETRAS ADL, CGI-S, CGI-I, and PGI C.

On Days 7, 21, 35, and 54 participants received a telephone call from site staff to ask about AEs and changes in concomitant medications.

After the DB part of the trial was complete, participants were able to choose to enter the Extension part of the trial. Participants who chose to enter the Extension began receiving OL study drug for up to 12 months, or start of the cross-over period, after a 43-day Extension DB Lead-in Period, whichever was earlier. During this Extension DB Lead-in Period, participants who received placebo or 60 mg in the DB part of the trial titrated up to 100 mg in a DB fashion utilizing the same dose titration regimen as in the DB part of the trial. Participants who received 100 mg in the DB part of the trial continued to receive 100 mg in a blinded fashion until all participants were receiving Compound 1 100 mg QAM as of Day 99. Participants who were unable to tolerate fixed-dose titration to 100 mg during the Extension DB Lead-in Period were discontinued from the trial and asked to attend the Safety Follow-up/EOS-E Visit. After the Extension DB Lead-in Period, participants flexibly dosed with Compound 1 (20-100 mg) QAM at the discretion of the Investigator during the Extension OL Period until the start of the cross-over period. Participants who consented to participate in the separate LTSS were able to end participation in the current study after completing the End of Treatment Visit and join the LTSS at any point during the Extension OL Period.

Those participants opting not to continue in the Extension proceeded directly to the Safety Follow-up Period from Day 57 to Day 70. The safety follow-up period lasted 14 days for participants in the DB part of the trial and lasted up to 14 days after the last dose of Compound 1 for participants in the Extension part of the trial. Participants who could not tolerate the Extension DB Lead-in Period titration dose in the Extension part of the trial were discontinued from the clinical trial as to not compromise the blinded nature of this Extension DB Lead-in Period, and they completed a safety follow-up visit.

Compound 1 was supplied as 5 mg and 20 mg modified-release tablets. Matching placebo was also supplied. Compound 1 was administered orally QAM with or without food and provided in pre-packaged containers to the participants. During the DB part of the study and the blinded lead-in period of the Extension, participants receiving Compound I received 6 tablets (a combination of Compound 1 and matching placebo tablets, depending on assigned dose level). Participants in the placebo group received 6 tablets of matching placebo. At the study visit on Day 1/Baseline, the study drug was dispensed from the Interactive Response Technology (IRT) and the in-clinic dose was taken from Kit A Day 1 at the visit. At subsequent study visits, the in-clinic dose was the last dose taken from the wallet card B dispensed at the prior study visit.

Compound 1 (20 mg strength) was provided to participants in OL bottles at the Day 99 clinic visit so open-label dosing could begin the following day (Day 100) for the start of the Extension open-label period. The tablet quantity varied depending on the dose level and formulation used.

At the re-randomization visit for the cross-over period, and every 7 days in that portion of the study, the treatment regimen was assigned by the IRT and dispensed by site pharmacist for direct-to-patient (DTP) shipping. In the cross-over period of the trial, all participants were centrally assigned to randomize to remain on the current regimen of Compound 1 or placebo using an IRT. After 3 weeks, participants receiving placebo were switched over to Compound 1 following the regimen described in Table 3. The IRT assigned treatment regimen based on the Tetras ADL response criteria after day 7.

In the DB part of the trial, eligible participants were randomized to receive 1 of 2 Compound 1 dosing regimens (60 mg or 100 mg) or placebo, administered orally QAM. Fixed titration regimens were used (Table 1). Participants were not allowed to adjust the number of tablets per day.

All participants who continued into the Extension part of the trial participated in a 43 day Extension DB Lead-in Period so all participants could be titrated to the 100-mg dose in a blinded fashion (Table 2) before receiving open-label, flexible doses of Compound 1 (20-100 mg) for up to 12 months (Days 100-459) during the Extension OL Period.

Selected items from the TETRAS-ADL and TETRAS-PS (or TETRAS-CUL, as appropriate) were scored in a modified manner and combined to create a TETRAS modified total score. Items 1-11 in the TETRAS ADL subscale and items 6 and 7 of the TETRAS-PS were used, with scoring adjusted so that response options 0 and 1 were combined into a single response of “0=Normal.” Further, scores of 2, 3, and 4 were adjusted to 1, 2, and 3, respectively. The TETRAS modified total score were the sum of these adjusted TETRAS-ADL and TETRAS PS items, having a possible range of 0 to 42, where larger values represent increased direct tremor impact to activities of daily living. The items that are included in the TETRAS-modified total score are illustrated in FIG. 2.

### Results

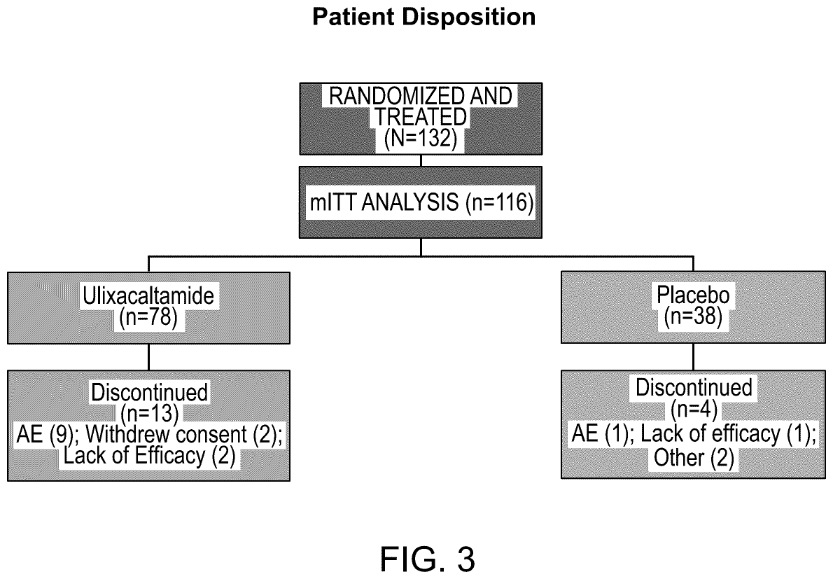

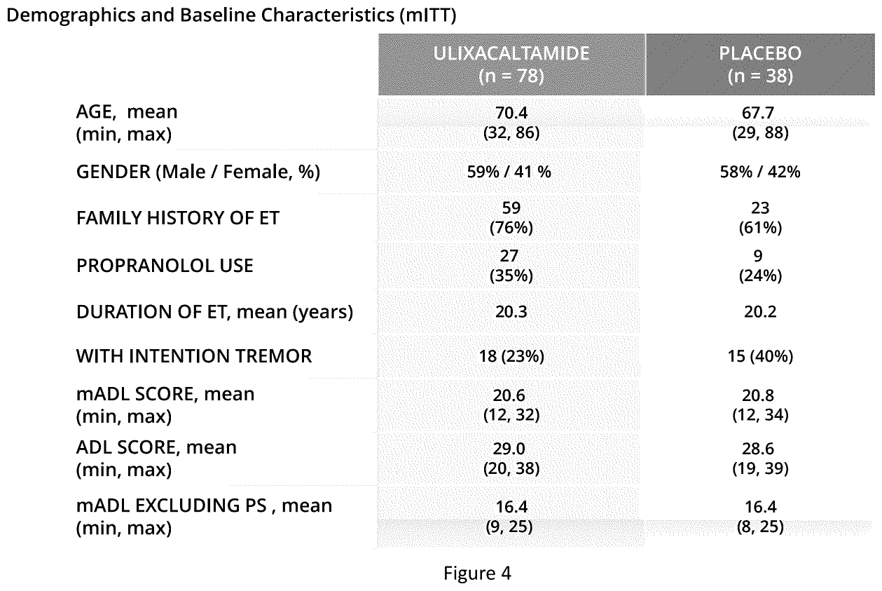

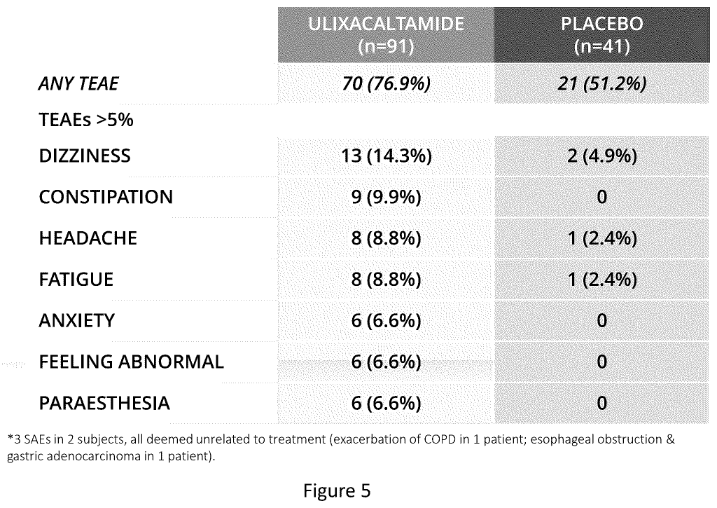

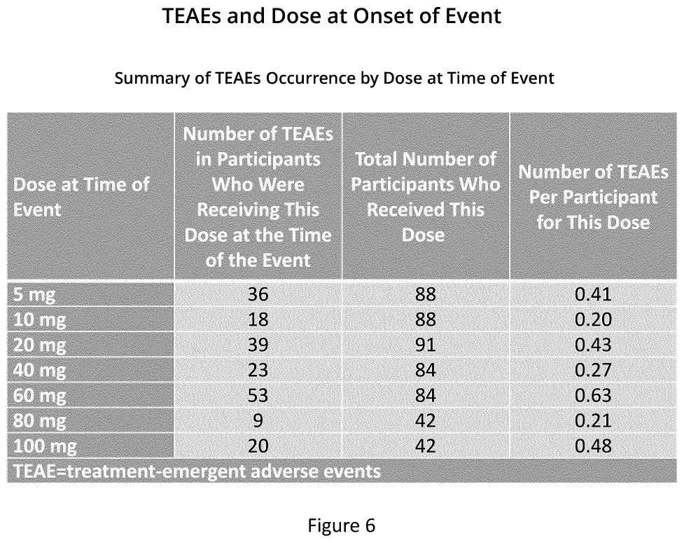

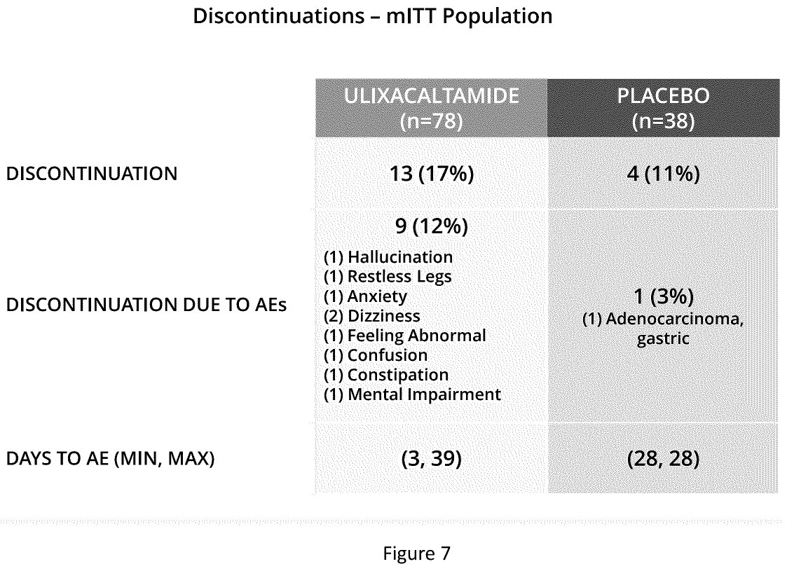

Compound 1 was well tolerated in the study, with no new safety findings. The majority of TEAEs were mild-moderate in severity, consistent with previous findings. There was a greater proportion of subjects who discontinued in the Compound 1 group at 17% vs in the placebo group with 11%. FIG. 3 is a schematic showing the patient disposition for the clinical trial. FIG. 4 is a table showing patient demographics and baseline characteristics (mITT). FIG. 5 is a table showing TEASs experienced by the patients in the clinical trial. FIG. 6 is a table showing TEAEs and the dose at onset of event. FIG. 7 is a table showing discontinuations of Compound 1 in the mITT patient population.

The primary efficacy measure in this clinical trial was change from baseline in mADL at day 56. Ulixacaltamide effect on individual mADL items was determined by calculating the fold-change adjusted by placebo change at Day 56 in mADL for individual mADL items. The secondary efficacy measures included change in Clinical Global Impression-Severity (CGI-S) score, Patient Global Impression-Change (PGI-C) score, TETRAS-ADL total score, TETRAS-UL score, TETRAS-CUL score and TETRAS-PS score.

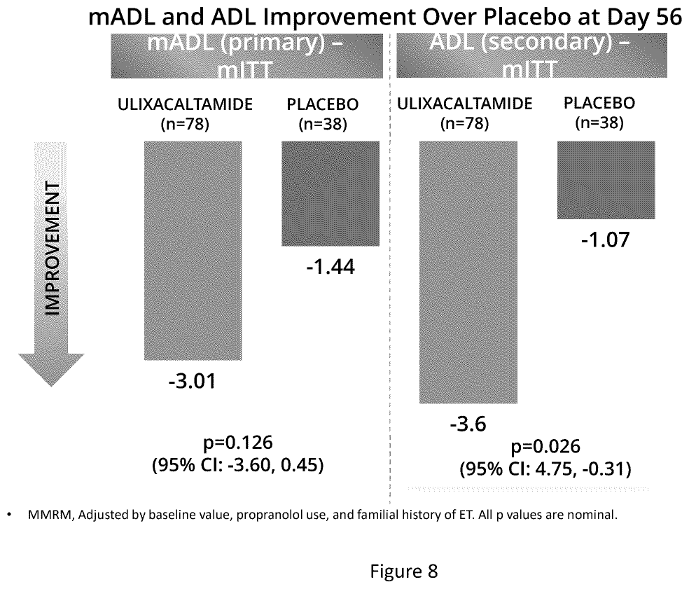

FIG. 8 is a bar graph showing change in mADL and ADL scores for mITT population treated with Compound 1 (ulixacaltamide) and placebo. As shown in FIG. 8, Compound 1 demonstrated a numerical difference vs. placebo in the mADL having a LS mean difference 1.57 and p=0.126. Participants treated with Compound 1 demonstrated mean improvement from baseline in ADL scores, with an average reduction of 3.6 points compared to baseline (n=78) and clinically and statistically different from the placebo group (n=38) (p−0.026) at day 56. Modified ADL scores in participants treated with Compound 1 (mean decrease of 2.69 points, n=78) also showed improvement over placebo (mean decrease of 0.88 points, n=38). For the secondary endpoint, change in TETRAS-ADL, nominal statistical significance was achieved having a [LS mean difference 2.53 and p=0.026].

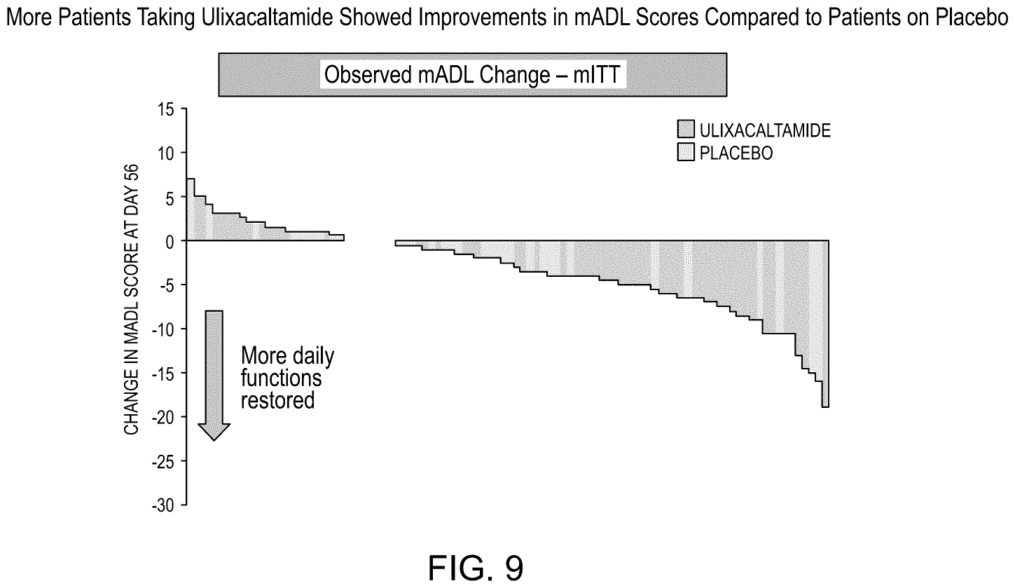

FIG. 9 is a bar graph showing change in mADL score at day 56 for patients who took ulixacaltamide (Compound 1) or placebo. FIG. 9 indicates that more patients taking ulixacaltamide showed improvements in mADL scores compared to patients taking placebo.

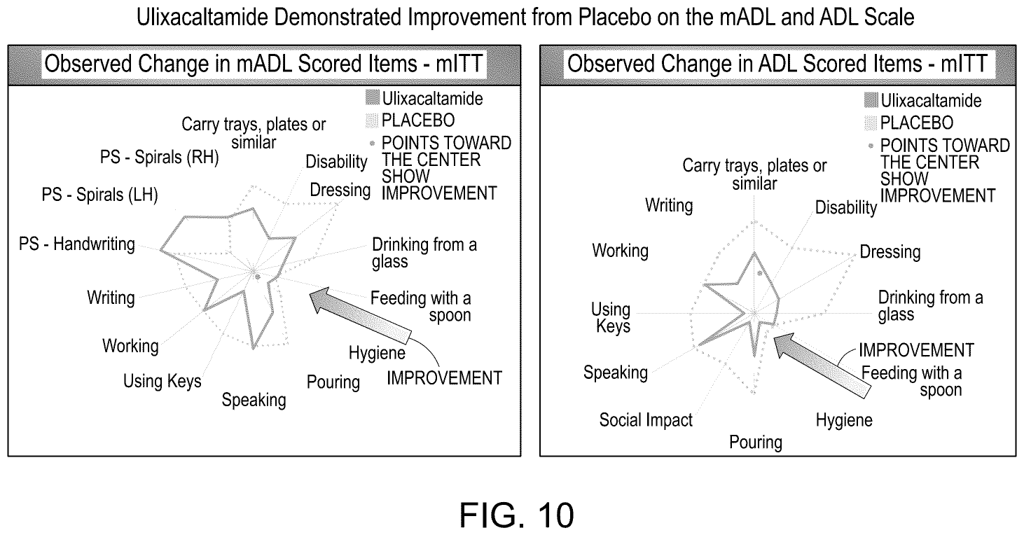

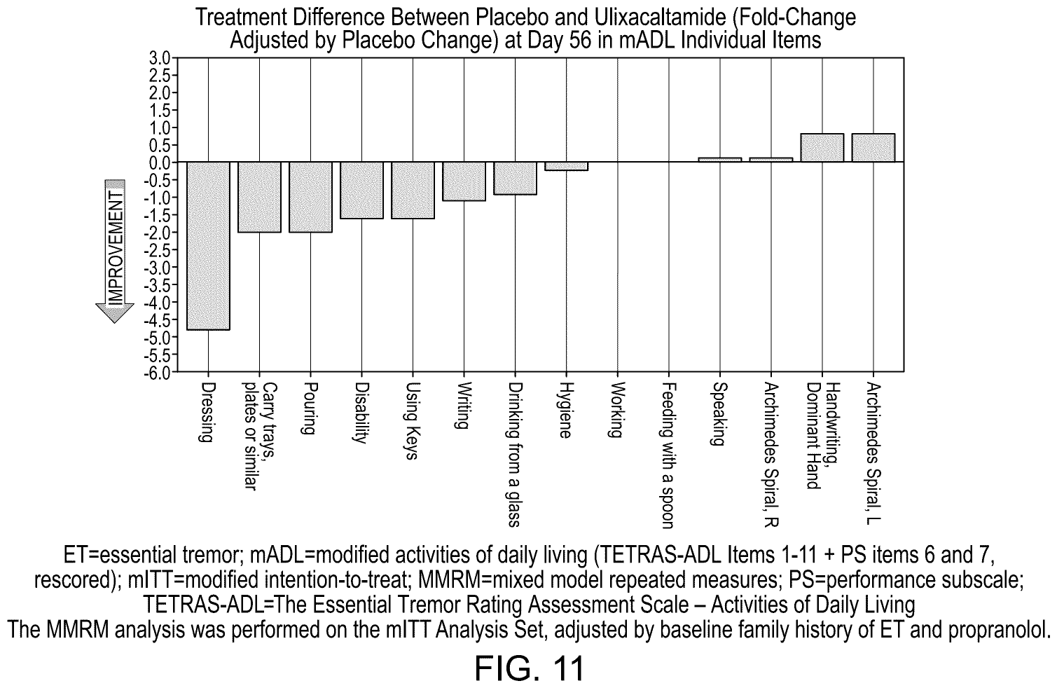

FIG. 10 is a schematic showing observed change in scores for individual items that were evaluated as a part of mADL and ADL assessment as compared to baseline. FIG. 11 is a bar graph showing fold-change adjusted by placebo change at day 56 for individual items that were evaluated as a part of mADL assessment. As shown in FIGS. 10 and 11, patients showed improvements in nearly all of the components of the ADL assessment, but showed little or no improvement in the additional components that were added to the mADL assessment. Ulixacaltamide demonstrated consistent effect relative to placebo for 8 of 11 ADL Scored Items. For the TETRAS PS items, dominant hand handwriting and left and right hand spirals, ulixacaltamide-treated patients demonstrated less improvement than those receiving placebo.

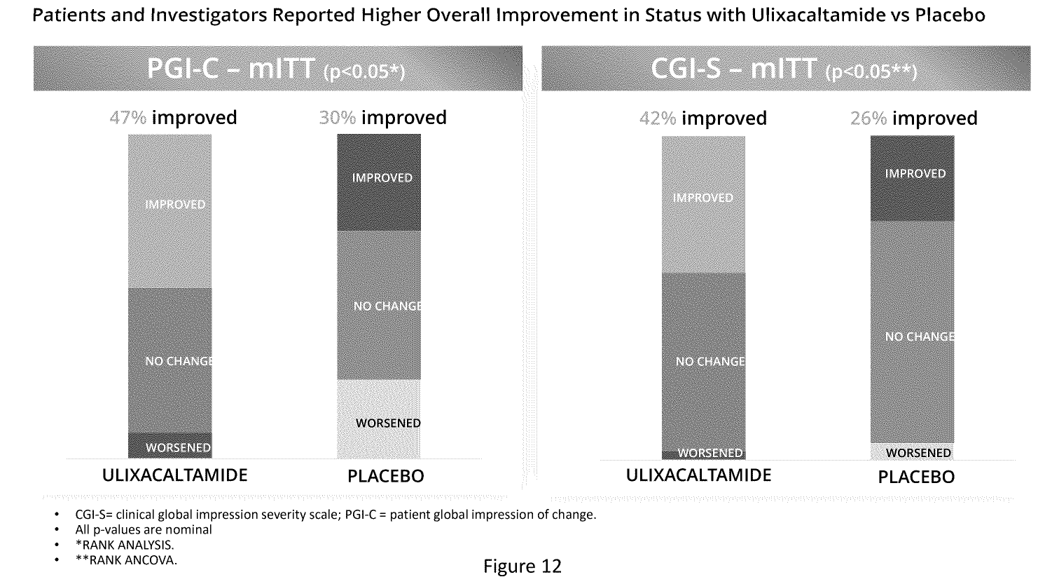

FIG. 12 shows graphs illustrating the status of patients in the mITT population measured using PGI-C scale and CGI-S scale. As shown in FIG. 12, patients and investigators reported higher overall improvement in status with Compound 1 compared to placebo using the PGI-C scale (47% improved) and CGI-S scale (42% improved). Nominal statistically significant improvements were observed in PGI-C and CGI-S.

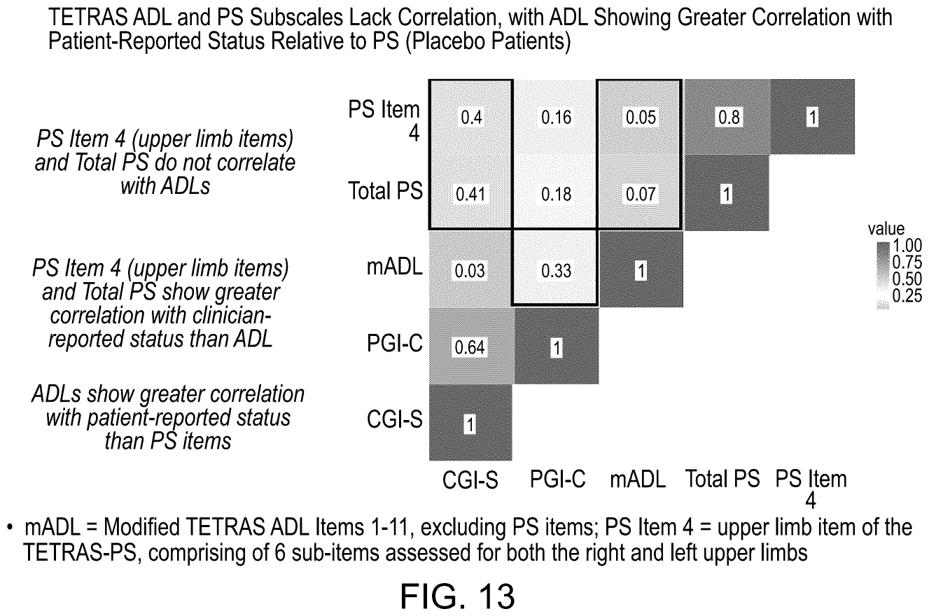

To assess the correlations between different endpoints in the study, Pearson's correlations were calculated for the change from baseline in the ADL and PS scores as well as other measures. Change from baseline in the total scores for subjects in the placebo arm were used to calculate r values for the five assessments shown. FIG. 13 is a schematic showing the results of the Pearson's correlation assessment for the change from baseline in the ADL and PS scores and other measures. The results shown in FIG. 13 are consistent with the pattern of clinical discordance between ADL and PS assessments. ADL showed no correlation with PS item #4 nor total PS. Interestingly, ADL Shows Greater Correlation with Patient-Reported Status Relative to PS, the PS assessments show greater correlation with clinician-reported status than does ADL.

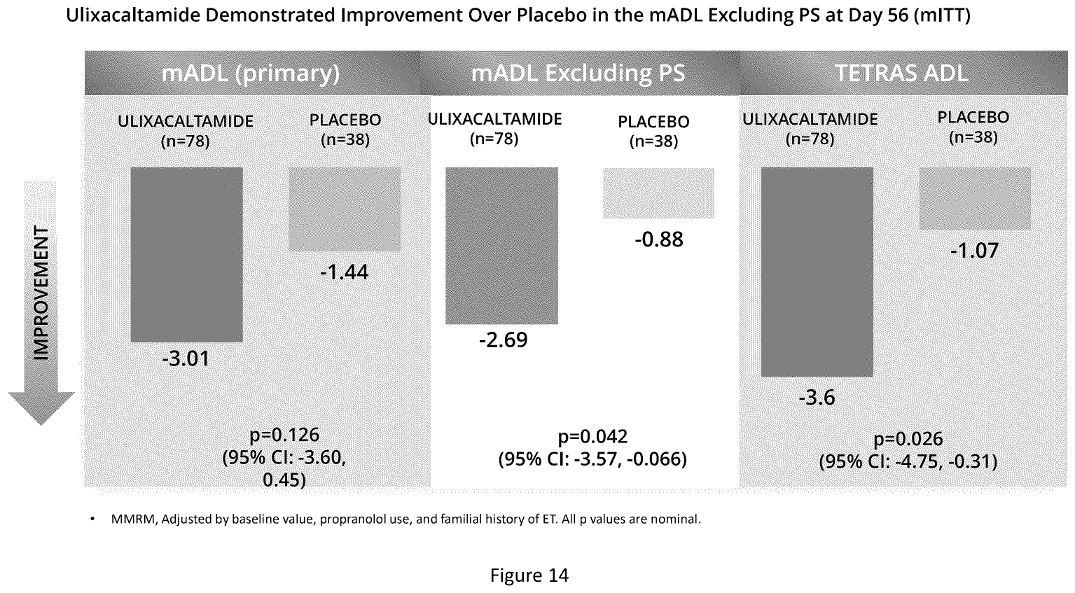

FIG. 14 is a bar graph showing change in mADL score at day 56 for each patient who took ulixacaltamide or placebo, when PS items (spirals—left and right, and handwriting) are excluded from assessment. The data from FIG. 8 is also included in FIG. 14 for comparison. As shown in FIG. 14, in a post hoc analysis, when excluding the PS items from the mADL, ulixacaltamide demonstrated nominally statistically significant improvement over placebo at Day 56 (mITT) [LS mean difference 1.81 and p=0.042].

In the post-hoc responder analysis using minimal clinically important difference distribution method, for the mITT population, patients taking ulixacaltamide were determined to have greater response rates compared to patients on placebo for the mADL and mADL excluding PS items. Given a 0.5 SD threshold, greater than 50% of ulixacaltamide treated participants had at least a 2 point improvement in their total score. When using a more stringent threshold of 1 SD, roughly 40% of ulixacaltamide treated subjects have at least a 4 point improvement in their total score.

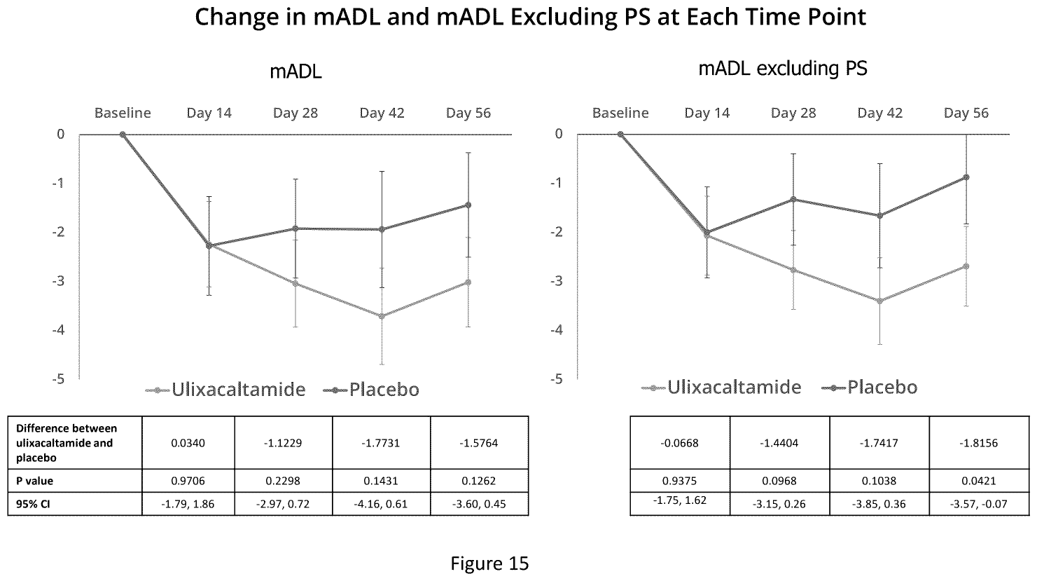

FIG. 15 is a graph showing change in mADL scores and mADL-excluding PS scores (mADL11) at each scored timepoint. FIG. 15 shows improvement in both mADL scores and mADL-excluding PS (mADL11) scores for ulixacaltamide vs. placebo after day 14.

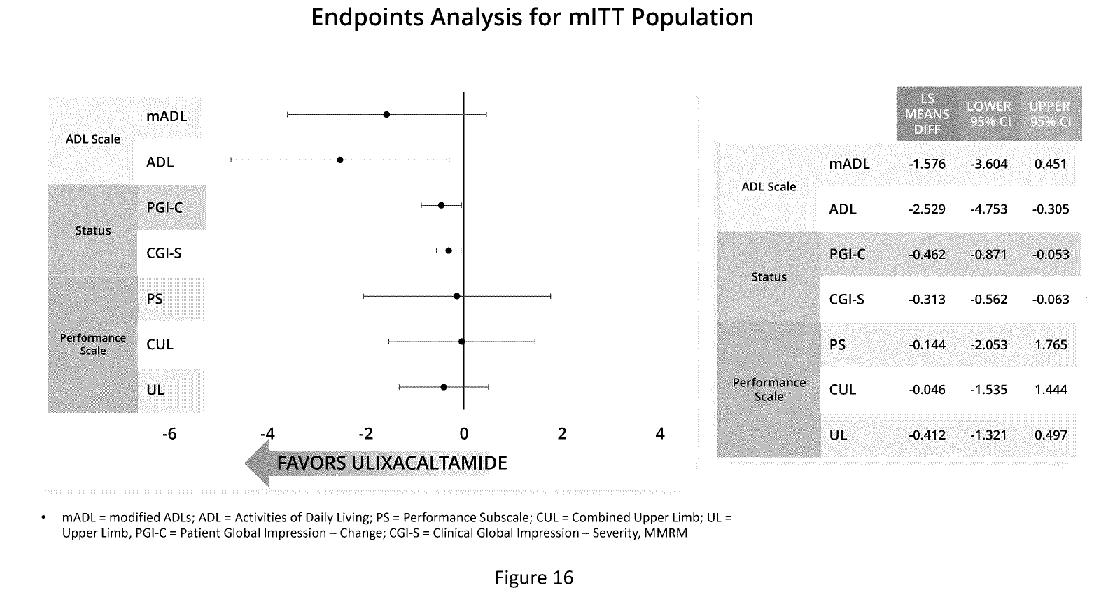

FIG. 16 is a graph and a table showing endpoints analysis for mITT population.

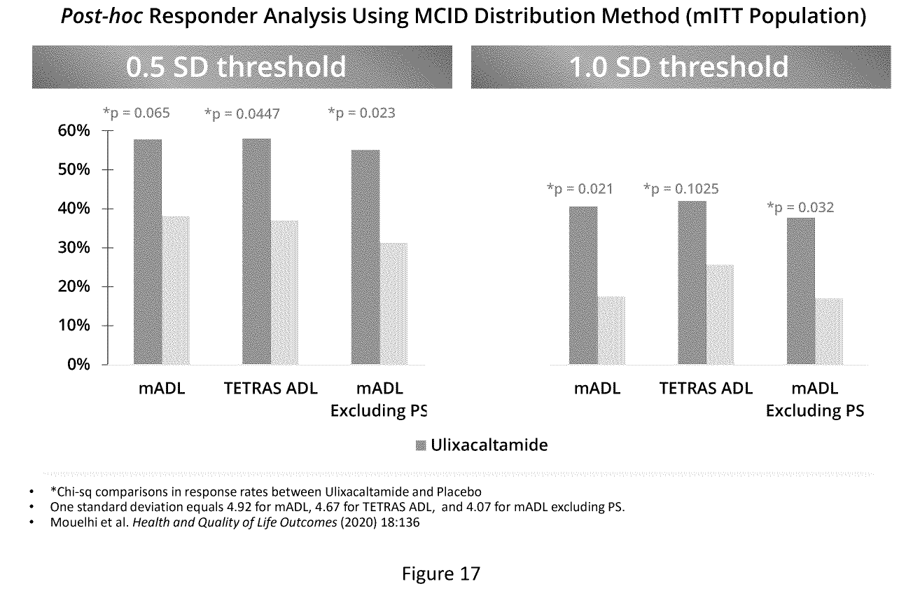

FIG. 17 is a bar graph showing the results of post-hoc responder analysis using MCID Distribution Method in mITT population. The results shown in FIG. 17 demonstrate that patients taking ulixacaltamide had greater response rates as compared to patients taking placebo as assessed by mADL score and mADL-excluding PS items score.

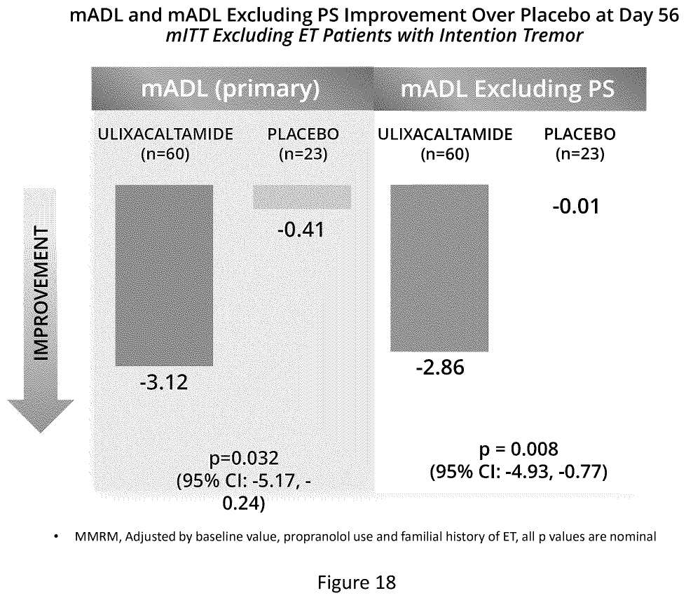

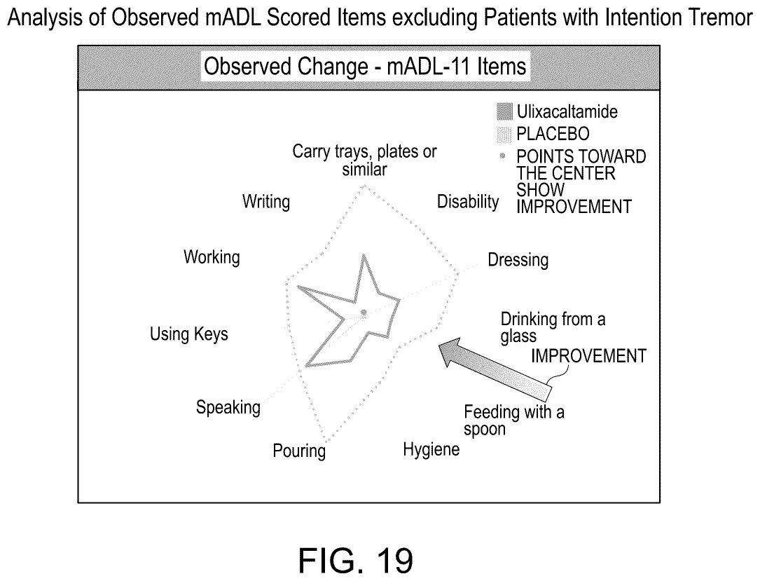

Patients who participated in the clinical trial included those with intention tremor. The effect of ulixacaltamide on the patient population that do not have intention tremor was analyzed. FIG. 18 is a bar graph showing mADL scores and mADL scores excluding PS in mITT population, excluding patients with intention tremor. FIG. 19 is a schematic showing observed change in scores for individual items that are evaluated as a part of mADL assessment as compared to baseline in patients who do not have intention tremor. FIGS. 18 and 19 demonstrate that there is a larger improvement in mADL-scored items as compared to baseline in patients with ET who do not have intention tremor.

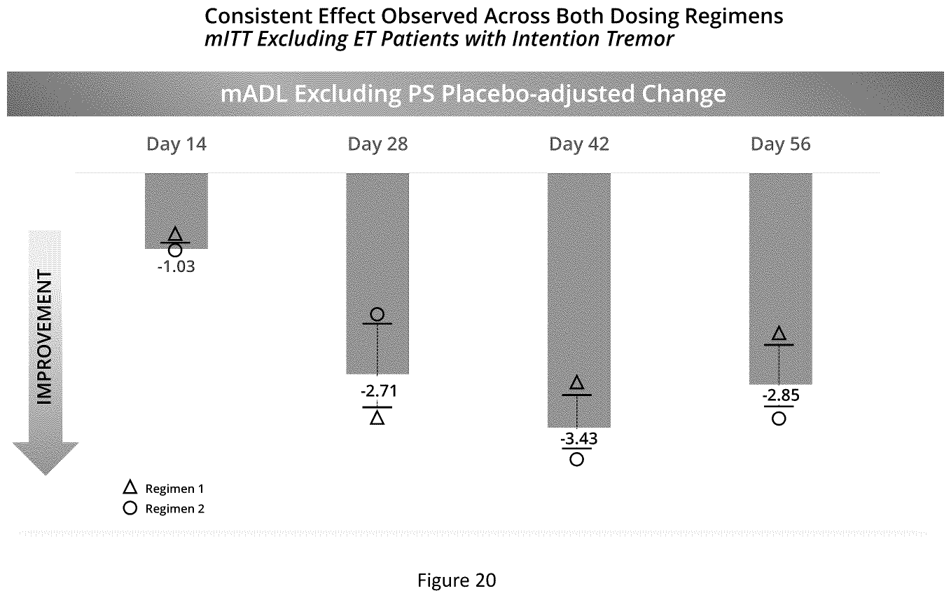

FIG. 20 is a bar graph showing change in placebo-adjusted mADL scores excluding PS in mITT patients without intention tremor for the two tested dosing regiments (60 mg and 100 mg). FIG. 20 demonstrates that there is a consistent effect observed across both tested dosing regimens in mITT patients without intention tremor.

Ulixacaltamide also demonstrated benefit to patients on propranolol. Specifically, 48% of patients on propranolol and ulixacaltamide in this Phase 2 trial achieved at least a 3-point improvement in mADL11 compared to 25% for patients on propranolol and placebo.

For the first time, modified TETRAS ADL items 1-11 (mADL11), excluding performance subscale (PS) items, have been identified as reliable and clinically meaningful measurements of treatment efficacy in ET. For example, distribution and anchor-based analysis methods have confirmed that a meaningful score difference (MSD) of about two or more points in mADL11 is an indicator of effective treatment of ET for a patient.

### Example 2. Open-Label Extension of the Phase 2 Trial

Following completion of the initial 8 weeks double-blind treatment phase of the Phase 2 trial described in Example 1, eligible patients had the option to continue their access to Compound 1 in an open-label extension phase (OLE). Participants who continued to the open label extension (OLE) phase remained blinded for a six-week lead-in phase.

In the Extension part of the trial, measures of efficacy include analysis of change from baseline to Days 70, 84, 99, 129, 159, 189, 279, 369, and 459 in modified ADL, CGI-S, TETRAS-ADL total score, TETRAS-PS total score, TETRAS UL score, TETRAS CUL score, and CGI-I and PGI-C scores at Days 70, 84, 99, 129, 159, 189, 279, 369, and 459.

There was no change to the overall safety results through 14 weeks of treatment with Compound 1.

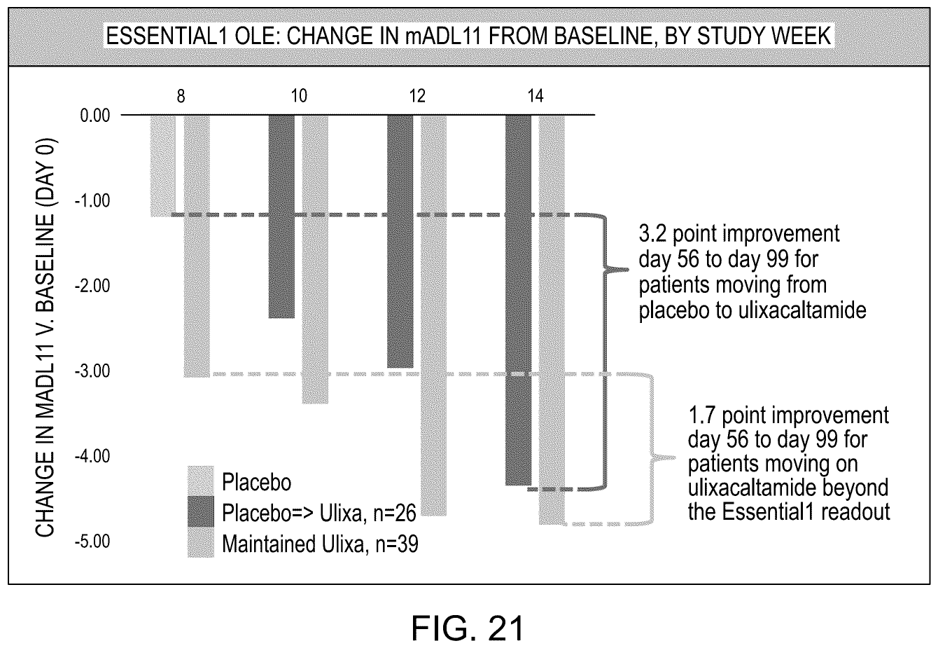

A total of 65 patients who completed the double-blinded portion of the Phase 2 trial described in Example 1 were eligible to participate in the OLE and completed the week 14 assessment. FIG. 21 is a bar graph showing change in mADL11 scores from baseline, by study week from week 8 to week 14. Patients who were eligible and continued on Compound 1 (n=39) experienced an additional mean improvement in mADL11 of 1.7 points from 3.09 at Weck 8 (95% CI: 0.98, 5.2) to 4.81 (95% CI: 2.38, 7.23) after 14 weeks of treatment. Patients who switched from placebo to Compound 1 treatment during the OLE 6-week lead-in (n=26) experienced mean improvement in mADL11 of 3.15 points from 1.21 at Week 8 (95% CI: −1.04, 3.46) to 4.36 (95% CI: 1.68, 7.05.

### Randomized Withdrawal Sub-Study

The open-label protocol was amended to further assess the criteria to be used in the upcoming randomized withdrawal study in Phase 3 (as described in Example 3). In this sub-study, patients were re-randomized in a blinded-fashion to either receive placebo or continue to receive Compound 1. Twenty-one patients who completed assessments at week 14 of the Open Label Extension were eligible to participate in the blinded sub-study.

Patients were evaluated weekly over a total of 6 weeks, with 11 patients assigned to Compound 1 and 10 patients assigned to placebo for the initial 3-week period, crossing over to either placebo or Compound 1 for an additional 3-week period. Blinded rescue was triggered for patients on placebo if loss in the mADL11 exceeded 2 points at any timepoint.

Patients who switched from Compound 1 to placebo experienced an average loss of effect in their mADL11 per week of 47% (mean loss of effect of −1.15 points/week), compared to 6% improvement in global mean change per week (mean improvement of 0.16 points/week) for the periods receiving Compound 1. In addition, 10 patients assigned to placebo met the rescue criteria to restart Compound 1.

85% of the patients who received Compound 1 (17 of 20) and 52% who received placebo maintained their mADL11 within 3-points compared to baseline, confirming the definition of patient stability to be used in the Phase 3 program. No new safety signals emerged and there was no change to the overall safety results observed in the 8-week double-blind treatment phase.

The results from the sub-study supported a number of proposed design elements for the upcoming Phase 3 randomized withdrawal study, including the responder criteria, and feasibility of rescuing patients with Compound 1.

### Example 3. Phase 3 Trial to Evaluate the Efficacy of the Compound of Formula (1) in the Treatment of Adults with Essential Tremor

### Overview

This Phase 3 study is a three-part, randomized, double-blind, placebo-controlled, fixed-dose (after titration) trial that will compare the efficacy and safety of Compound 1 with placebo in participants aged 18 to 80 years who have a diagnosis of ET. The primary objective of Study 1 parallel design (PD) is to evaluate the change from Baseline in mADL11 score for Compound 1 compared to placebo after 12 weeks (84 days) of treatment. The primary objective of Study 2 randomized withdrawal (RW) is to evaluate the maintenance of response by measuring the proportion of participants that maintain response following RW. Other efficacy assessments in Studies 1 and 2 include Clinical Global Impression-Severity (CGI-S), Patient Global Impression-Severity (PGI-S), Patient Global Impression-Change (PGI-C), and Archimedes spiral drawing. The primary objective of Study 3 long-term safety study (LTSS) is to assess the long-term safety of up to approximately 1 year of Compound 1 treatment. The safety and tolerability of Compound I will be assessed throughout all 3 studies.

This is a decentralized Phase 3 clinical trial, utilizing a combination of in-home and telehealth visits, and consisting of 3 simultaneous studies that include a randomized, DB, placebo-controlled PD study, a DB RW study, and an LTSS study. The studies in this trial are designed to evaluate the efficacy and/or safety of Compound 1 in participants aged 18 to 80 years who have a diagnosis of ET and have had symptoms for at least 3 years. Study 1 PD and Study 2 RW participants will be informed of all 3 studies and must agree during informed consent that, if deemed eligible, they will be assigned to either Study 1 PD or Study 2 RW and rollover into Study 3 LTSS. A specific informed consent form (ICF) will be used for Phase 2 OLE participants that rollover into Study 3 LTSS.
In Study 1 PD, participants will receive 12 weeks (84 days) of either Compound 1 or matching placebo in a DB fashion.
In Study 2 RW, participants will receive 8 weeks (56 days) of Compound 1 in a DB fashion before entering a 4-week DB RW period, where responders will undergo RW and non-responders will continue Compound I treatment. Responder participants in the RW period who then meet the predefined relapse criteria will rollover into Study 3 LTSS with blinded dose titration as described in Table 6.
In Study 3 LTSS participants will receive up to a total of approximately 1 year of Compound 1, which includes prior study dosing.

Compound I will be titrated to 60 mg over a 2-week period (Table 8). After the baseline visit, participants will administer either Compound 1 or placebo every morning (QAM) at home prior to their scheduled home health or telehealth visits, as applicable. Dose adjustments will not be allowed. Participants who complete Studies 1 PD or Study 2 RW (complete all assessments through Day 84), meet the predefined relapse criteria in Study 2 RW, or complete a Phase 2 EOT visit may rollover into Study 3 LTSS, with Day 84 assessments carried forward from their respective studies.

The duration of Studies 1 PD and 2 RW can be as long as 18 weeks, including the Screening Period (Table 4) and the Treatment and Safety Follow-up (SFU) Periods (Table 5 and Table 6). The duration of the LTSS will be up to approximately 1 year (Table 7).

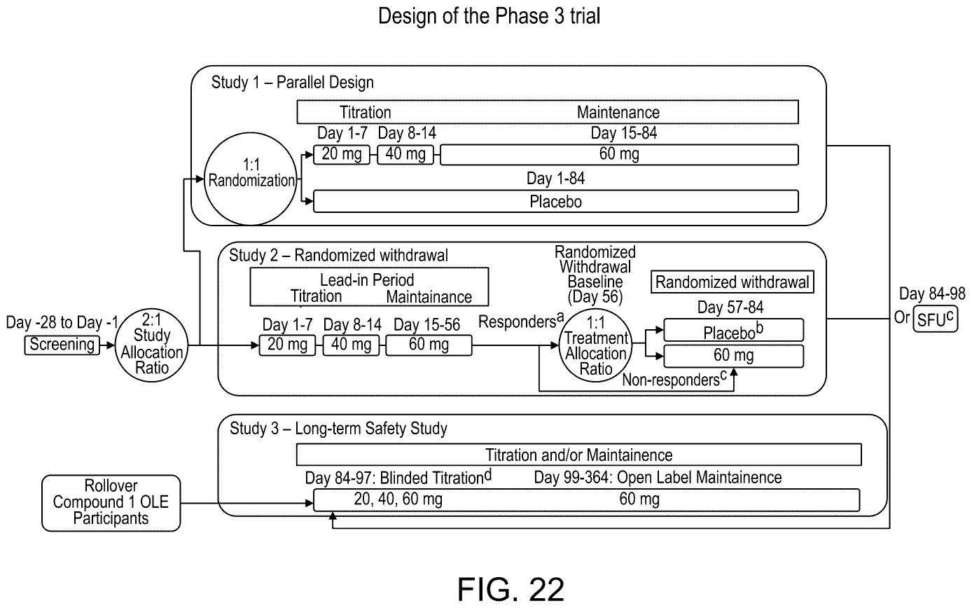

FIG. 22 is a schematic showing the design of the Phase 3 trial.

TABLE 4
Pivotal Studies: Screening Period

| Study Period | Description |
|---|---|
| Screening | 4 weeks with weekly visits as described below: |
| Period | Home Health Visit: Day −21 |
| | Telehealth Visits: Day −28, Day −21, Day −14, and Day −7 |

TABLE 5
Study 1 PD: Intervention and Safety-Follow Up Periods

| Study Period | Description |
|---|---|
| Baseline Visit | Day 1: Home Health Visit and Telehealth Visit |
| Treatment Period | 12-week duration |
| | Titration Period: 2 weeks |
| | Week 1 (Days 1 through 7) 20 mg QAM or placebo |
| | Week 2 (Days 8 through 14) 40 mg QAM or placebo |
| | Maintenance Period: 10 weeks |
| | Week 3 to Week 12 (Days 15 through 84) 60 mg QAM |
| | or placebo |
| Safety Follow-up Period | 2 weeks (14 days) following the last dose of study drug for those |
| | who either complete the study and choose not to participate in |
| | Study 3 LTSS or discontinue treatment early for any reason. |

Abbreviations: LTSS = long-term safety study; QAM = every morning.

TABLE 6
Study 2 RW: Intervention and Safety Follow-Up Periods

| Study Period | Description |
|---|---|
| Baseline Visit | Day 1: Home Health Visit and Telehealth Visit |
| DB Intervention Period: | 8-week duration |
| RW Lead-in Period | Titration Period: 2 weeks |
| | Week 1 (Days 1 through 7) 20 mg QAM |
| | Week 2 (Days 8 through 14) 40 mg QAM |
| | Maintenance Period: 6 weeks |
| | Week 3 to Week 8 (Days 15 through 56) 60 mg QAM |
| DB Intervention Period: | 4-week duration |
| RW Period | Randomized Compound 1 Responder Cohort: Randomized |
| | 1:1 to either continue Compound 1 treatment at their current |
| | dose (60 mg) or transition to placebo. |
| | Relapse Group: Transfer to Study 3 LTSS and either |
| | maintain Compound 1 or begin 2-week titration from |
| | placebo to 60 mg. |
| | Nonrandomized Cohort: Remain on Compound 1 60 mg |
| Safety Follow-up Period | 2 weeks (14 days) following the last dose of study drug for those |
| | who either complete the study and choose not to participate in |
| | Study 3 LTSS or discontinue treatment early for any reason. |

Abbreviations: DB = double blind; LTSS = long-term safety study; QAM = every morning; RW = randomized withdrawal.

TABLE 7
Study 3 LTSS: Intervention and Safety Follow-Up Periods

| Study Period | Description |
|---|---|
| LTSS Baseline | Day 84: Telehealth Visit for Phase 2 OLE participants, after |
| | informed consent and investigator review of most recent OLE |
| | safety assessments. |
| | Study 1 and 2 participants that rollover into LTSS will enter at Day |
| | 84.^{a} |
| Intervention Period | Up to approximately 1 year in duration, with DB administration of |
| | Compound 1 through Week 14. |
| | Blinded Titration Period^{b}: 2 weeks |
| | Days 84 through 90: 20 mg QAM (or maintain 60 mg) |
| | Days 91 through 97: 40 mg QAM (or maintain 60 mg) |
| | Maintenance Period: 50 weeks |
| | Day 99 up to approximately 1 year: 60 mg QAM |
| Safety Follow-up Period | 2 weeks (14 days) following the last dose of study drug for those |
| | who either complete the study and choose not to continue in Study |
| | 3 LTSS or discontinue treatment early for any reason. |

Abbreviations: DB = double blind; EOT = end of treatment; LTSS = long-term safety study; OLE = open label extension; PD = parallel design; QAM = every morning; RW = randomized withdrawal.
^{a}For rollover participants, Day 84/EOT assessments should be carried over from Study 1 PD or Study 2 RW as the LTSS baseline.
^{b}A blinded titration period will occur during the first two weeks of Study LTSS for participants rolling over from Studies 1 and 2. During this blinded titration period, participants will either titrate from their placebo dose to 60 mg over a 2-week period or maintain their 60 mg dose.

Eligible participants will be randomized to receive oral Compound I or placebo QAM. Fixed titration regimens will be used. as described in Table 8.

Dose adjustments will not be allowed.

TABLE 8
Intervention Period Dosing Sequence by Treatment Group

| | Intervention Period | | |
|---|---|---|---|
| Study | Titration | | Maintenance |
| Study 1 PD | Week 1 | Week 2 | Week 3 to Week 12 |
| Study 2 RW | (7 days) | (7 days) | (70 days) |

| | |
|---|---|
| Lead-in Period | Randomized Withdrawal |
| Week 3 to | Week 9 to |
| Week 8 | Week 12 ^{a} |
| (42 days) | (28 days) |

| Study 3 | Week 1 | Week 2 | Maintenance |
|---|---|---|---|
| LTSS | (7 days) ^{b} | (7 days) ^{b} | Week 3^{c} up to 1 Year |
| Treatment | Dose | | |
| Compound 1 | 20 mg | 40 mg | 60 mg |

| Placebo | Matching Placebo |
|---|---|
| Study | Duration |
| Study 1 PD | 84 Days |
| Study 2 RW | 84 Days |
| Study 3 LTSS | Up to approximately 1 Year |

LTSS = long term safety study; OLE = open label extension; PD = parallel design; RW = randomized withdrawal.
^{a} In Study 2 RW, after 6 weeks of maintenance dosing, participants that meet the responder criteria will receive either 4 weeks of placebo or ulixacaltamide. The remainder of participants will remain on ulixacaltamide.
^{b} Only for participants who are not currently on ulixacaltamide (i.e., are on placebo in Study 1 PD or Study 2 RW prior to entering Study 3 LTSS). For participants rolling over from Study1 PD and Study 2 RW, dosing over the first 2 weeks of the LTSS study will remain blinded.
^{c}Participants entering Study 3 LTSS after completing Study 1 PD or Study 2 RW will remain on a 60 mg dose or begin titration of ulixacaltamide to 60 mg as defined in the schedule above. Participants entering Study 3 LTSS from PRAX-944-222 OLE will remain on 60 mg or titrate over a two-week period to 60 mg as follows: if their current dose is greater than 60 mg they will reduce their dose directly to 60 mg and if lower than 60 mg they will titrate based on the titration schedule described above, with a dose increase every 7 days based on their current dose (i.e. 20 or 40 mg).

The objectives and endpoints for Study I PD are described below (Table 9).

TABLE 9
Objectives and Endpoints for Study 1 PD

| | |
|---|---|
| Objectives | Endpoints |

Primary
Efficacy

| | |
|---|---|
| To evaluate the efficacy of Compound 1 | Change from Baseline to Day 84 on the |
| compared with placebo in participants | mADL11 |
| with ET | |

Secondary
Efficacy

| | |
|---|---|
| To further evaluate the efficacy of | Proportion of participants responding |
| Compound 1 compared with placebo in | to ulixacaltamide, as defined by |
| participants with ET over time | change in mADL11 score after 12 |
| | weeks (84 days) of treatment |
| | Change from Baseline to Day 84 in: |
| | TETRAS-ADL |
| | PGI-C |
| | CGI-S |
| | PGI-S |
| | Proportion of participants responding |
| | to ulixacaltamide, as defined by |
| | change in mADL11 score after 14, 28, |
| | 56, and 70 days of treatment |
| | Change from Baseline to Day 14, Day |
| | 28, Day 56, and Day 70 in: |
| | mADL11 |
| | TETRAS-ADL |
| | PGI-C |
| | CGI-S |
| | PGI-S |
| | Dominant Hand Archimedes Spiral |
| | (Day 1, Day 56, Day 84) |

Safety

| | |
|---|---|
| To evaluate the safety of Compound 1 | Incidence and severity of AEs, including |
| compared to placebo in participants with | discontinuation of study drug due to AEs |
| ET | Changes in vital sign measurements |
| | Changes in clinical laboratory results |
| | Changes in ECG parameters |
| | Incidence of C-SSRS measured suicidal |
| | ideation or behavior |
| | Changes in BDI-II and BAI |

Exploratory
Clinical Pharmacology

| | |
|---|---|
| To determine plasma concentrations of | Plasma Compound 1 concentrations |
| Compound 1 in participants with ET | |

AE = adverse events; BAI = Beck Anxiety Index; BDI-II = Beck Depression Inventory - Second Edition; CGI-S = Clinical Global Impressions-Severity; C-SSRS = Columbia-Suicide Severity Rating Scale; ECG = electrocardiogram; ET = essential tremor; mADL11 = modified TETRAS-ADL items 1 to 11 with modified score; PGI-C = Patient Global Impression of Change; PGI-S = Patient Global Impression of Severity; TETRAS-ADL = The Essential Tremor Rating Assessment Scale - Activities of Daily Living.

TABLE 10
Objectives and Endpoints for Study 2 RW

| | |
|---|---|
| Objectives | Endpoints |

Primary
Efficacy

| | |
|---|---|
| To evaluate Compound 1 efficacy | The proportion of participants that |
| maintenance of response in the RW | maintain response, as defined by change |
| portion of the study | in mADL11 score, following RW |

Secondary
Efficacy

| | |
|---|---|
| To further evaluate the efficacy of | Change from RW Baseline (Day 56) to |
| continued Compound 1 over time | Day 63, Day 70, Day 77, and Day 84 on |
| | the following: |
| | mADL11 |
| | TETRAS-ADL |
| | PGI-C |
| | CGI-S |
| | PGI-S |
| | Dominant hand Archimedes Spiral |
| | (Day 1, Day 56, Day 84) |

Safety

| | |
|---|---|
| To evaluate the safety of Compound 1 in | Incidence and severity of AEs, |
| participants with ET | including discontinuation of study drug |
| | due to AEs |
| | Changes in vital sign measurements |
| | Changes in clinical laboratory results |
| | Changes in ECG parameters |
| | Incidence of C-SSRS measured suicidal |
| | ideation or behavior |
| | Changes in BDI-II and BAI |

Exploratory
Clinical Pharmacology

| | |
|---|---|
| To determine plasma concentrations of | Plasma Compound 1 concentrations |
| Compound 1 in participants with ET | |

AE = adverse events; BAI = Beck Anxiety Index; BDI-II = Beck Depression Inventory - Second Edition; CGI-S = Clinical Global Impression of Severity; CSSRS = Columbia-Suicide Severity Rating Scale; ECG = electrocardiogram; ET = essential tremor; mADL11 = modified TETRAS-ADL items 1 to 11 with modified score; PGI-C = Patient Global Impression of Change; PGI-S = Patient Global Impression of Severity; RW = randomized withdrawal; TETRAS-ADL = The Essential Tremor Rating Assessment Scale-Activities of Daily Living.

The objectives and endpoints for Study 3 LTSS are described below (Table 11).

TABLE 11
Objectives and Endpoints for Study 3 LTSS

| | |
|---|---|
| Objective | Endpoints |

Primary

| | |
|---|---|
| To evaluate the long-term | Incidence and severity of AEs |
| safety of Compound 1 in | Changes in vital sign measurements |
| participants with ET | Changes in clinical laboratory results |
| | Changes in ECG parameters |
| | Incidence of C-SSRS measured |
| | suicidal ideation or behavior |

AE = adverse events; C-SSRS = Columbia-Suicide Severity Rating Scale; ECG = electrocardiogram; ET = essential tremor.

### Background and Clinical Trial Rationale

Compound 1 is a novel small molecule being developed for the treatment of essential tremor (ET) that is a high-affinity, state-dependent, selective inhibitor of T-type calcium ion (Ca₂₊) channels. Comprised of 3 isoforms (CaV3.1, CaV3.2, and CaV3.3), T-type Ca²⁺ channels are expressed widely throughout the brain, especially in the cerebello-thalamo-cortical (CTC) circuit. Aberrant bursts in the CTC circuit occur at the same frequency as upper limb tremor in ET.

ET is the most common adult movement disorder, affecting up to 2% of the United States (US) population with an estimated true prevalence of approximately 7 million Americans. ET is characterized by a 6 to 12 Hz postural and kinetic tremor (i.e., tremor during voluntary movement) in the upper limbs. The most characteristic clinical feature is kinetic tremor of the arms and hands, but tremor may also occur in the head and voice as the disease progresses and, less commonly, in the face, legs, and trunk. Emerging evidence supports the understanding that ET represents a heterogeneous syndrome and diagnosis requires further differentiation based on medical history and neurological exam.

ET has a significant impact on several dimensions of daily functioning in patients, especially writing, eating, and drinking. Functional disability was reported in over 70% of patients with ET, and was associated with comorbid anxiety and depression.

Despite being one of the most common movement disorders, ET often remains undertreated or untreated, in part because of the limited number of available treatments. Propranolol and primidone are the medications used most frequently to treat ET. Propranolol is the only FDA-approved orally administered treatment indicated for the treatment of ET. Propranolol was originally developed and approved for hypertension in 1967 and was approved for the treatment of ET in 1986 on the basis of a 2-week clinical trial that enrolled only 9 patients and evaluated a limited dose range of 40 mg to 80 mg three-times daily. As scientific understanding of ET has matured and clinical experience with propranolol has been gained, propranolol has emerged as an inadequate, poorly tolerated treatment, leaving a severe unmet medical need for patients with ET. Propranolol in the treatment of ET exhibits limited efficacy with side effects (e.g., bradycardia) that often lead to discontinuation. The American Academy of Neurology 2011 ET treatment guideline states that not all patients improve on or tolerate primidone and propranolol. Our most recent data indicate that for patients who are receiving pharmacotherapy, over 40% discontinue all pharmacological options after 2 years of treatment. Thus, while there is at least 1 approved therapy, the currently available medications for ET fail to meet the needs of most patients.

The results of the Phase 2 trials with ulixacaltamie (see Examples 1 and 2) support the efficacy and safety of ulixacaltamide in patients with ET. The Phase 2 trials demonstrated reduced modified ADL (mADL) scores (i.e., reduced tremor symptoms) at daily doses ranging from 20 to 120 mg for up to 56 days. In the double-blind (DB) portion of the Phase 2 study, the rescored TETRAS-ADL items 1-11 (mADL11) score decreased more in participants randomized to Compound 1 compared with placebo after 56 days of dosing, with least square mean (LSM standard error [SE]) scores of (2.69 [0.81]) compared with placebo (−0.87 [0.95]). The LSM (95% confidence interval [CI]) treatment difference was −1.82 (95% CI: −3.565, −0.066; with a nominal p value of <0.05). In the Phase 2 extension period, 20 participants completed a blinded 6-week crossover period. Participants were randomized in blinded fashion to either 3 weeks of Compound 1 60 mg or placebo. Rescue treatment (Compound 1) was offered to participants if their mADL11 worsened by a preset threshold. During the crossover period, maintenance of response was observed in 17 of 20 participants receiving Compound 1 compared to 11 of 21 participants receiving placebo, with a Chi-squared p-value of 0.0249 and 0.0388 when adjusted for propranolol use, family history and intention tremor (IT). None of the Compound 1-treated participants and 9 of 21 placebo-treated participants qualified for and received rescue treatment within the 3-week period. No new safety signal was identified during this portion of the study.

Across the clinical development program, Compound 1 has been generally well tolerated following up to 129-days of daily administration at doses ranging from 5 to 120 mg, demonstrating a favorable safety profile following a titration schedule. No deaths or serious adverse events (SAEs) considered by the sponsor as being related to study drug have been reported following administration of Compound 1. Likewise, no suicide attempts or pregnancies have been reported following administration of Compound 1.

This Phase 3 study is a three-part, randomized, DB, placebo-controlled, fixed-dose (after titration) trial that will compare the efficacy and safety of Compound 1 with placebo in participants aged 18 to 80 years who have a diagnosis of ET. The primary objective of Study 1 parallel design (PD) is to evaluate the change from Baseline in mADL11 score for Compound 1 compared to placebo after 12 weeks (84 days) of treatment. The primary objective of Study 2 randomized withdrawal (RW) is to evaluate the maintenance of response by measuring the proportion of participants that maintain response following RW. Other efficacy assessments in Studies 1 and 2 include Clinical Global Impressions-Severity (CGI-S), Patient Global Impression-Severity (PGI-S), Patient Global Impression-Change (PGI-C) and Archimedes spiral drawing. The primary objective of Study 3 long-term safety study (LTSS) is to assess the long-term safety of up to approximately 1 year of Compound 1 treatment. The safety and tolerability of ulixacaltamide will be assessed throughout all 3 studies.

### Overall Design

This trial is a decentralized, Phase 3, multi-study, clinical trial evaluating the safety and efficacy of Compound 1 in ET. The trial includes 2 separate simultaneous pivotal phase 3 studies with all participants undergoing one screening process. This simultaneous study design promotes a more homogeneous study population and ensures blinded study assignment and treatment arm allocation across Study 1 PD and Study 2 RW, an objective that could not be met if Study 2 RW were conducted in isolation.

The trial consists of 3 simultaneous, decentralized, studies including 2 pivotal DB placebo-controlled studies and an LTSS in participants with ET as follows:

Study 1 PD: A 12-week PD, randomized, DB, placebo-controlled study to evaluate the change from Baseline in mADL11 score for Compound 1 compared to placebo after 12 weeks (84 days) of treatment.

Study 2 RW: A 12-week RW, DB, placebo-controlled study to evaluate the efficacy and maintenance of response and durability of effect in participants who respond to Compound 1 and safety.

Study 3 LTSS: An LTSS to evaluate the long-term safety of Compound 1 for up to approximately 1 year. The first 2 weeks of the LTSS will include a 2-week blinded titration period for participants who rollover from Study 1 PD and Study 2 RW to maintain the blind for these studies. The remainder of the study will be open label. Participants who are enrolled in the Phase 2 open label extension (OLE) can rollover to Study 3 LTSS and will either begin titration over a 2-week period to 60 mg or continue taking the 60 mg dose.

Both pivotal studies include a Screening Period and all 3 studies have Intervention and Safety Follow-up Periods. There are two types of study visits: home health visits and telehealth visits. Home health visits will be staffed by a trained research nurse traveling to the participant's home. The investigator and/or research staff will participate in home health visits remotely with audiovisual communication. The telehealth visit will be a remote audiovisual visit with the investigator and/or research staff. Investigator will supervise all visits. In addition to home health and telehealth visits, study drug will be directly mailed to participants.

### Screening and Baseline Periods

Key screening assessments include medical history, demographics, prior and concomitant medications, physical and neurologic examination, drug/alcohol screen, pregnancy screen, clinical laboratory evaluations, electrocardiogram (ECG), vital signs, Columbia-Suicide Severity Rating Scale (C-SSRS), and assessments of ET severity using The Essential Tremor Rating Assessment Scale-Activities of Daily Living (TETRAS-ADL).

The Screening Period for Compound 1 naïve participants will be 28 days in duration. After all screening assessments are completed and a participant is deemed eligible for the study, randomization will occur between Day −7 and Day −3 to allow time for assigned study drug delivery to the participant's home.

Participants will complete the TETRAS-ADL assessment 3 times during Screening (on Day −28, Day −14, and Day −7).

Phase 2 OLE participants will not be required to complete this screening process, but the investigator must review the most recent safety assessments for these participants approximately 1 week before their first LTSS study visit. If safety issues are identified, the investigator will review with the sponsor to determine if participant is eligible to continue Compound 1 in the LTSS. Throughout the Intervention Periods in Studies 1 PD, 2 RW, or 3 LTSS, participants who choose or are required to stop study drug dosing must have an early discontinuation (ED) visit scheduled as soon as possible. If participants choose not to participate in the Study 3 LTSS after completion of Study 1 PD or Study 2 RW, they must enter the 14-day Safety Follow-up Period. Participants who prematurely discontinue from Study 1 PD or Study 2 RW will not be eligible to participate in the LTSS.

Participants will self-administer either ulixacaltamide or placebo every morning (QAM) at home prior to their scheduled home health or telehealth visits, as applicable. Key efficacy assessments will be performed at select visits based on study assignment and include TETRAS-ADL, PGI-C, PGI-S, CGI-S, and Archimedes spiral drawing. Drug concentration samples (on Day 56 and Day 70) will be drawn at the same time as clinical laboratory assessments, following the correct collection order.

### Study 1 PD

All participants assigned to Study 1 PD who continue to meet all clinical trial entry criteria on Day I will receive DB treatment with Compound 1 or placebo from Day 1 through Day 84, using a fixed-dose regimen (following titration).

On Baseline (Day 1) participants will receive 1 tablet of study drug during the home health visit and then self-administer QAM from Day 2 to Day 84. Assessments for CGI-S, PGI-S, and Archimedes Spiral will be performed in that order before dosing.

For participants randomized to Compound 1, the titration period will consist of a 20 mg QAM dose during Week 1 (Days 1 to 7) and a 40 mg QAM dose during Week 2 (Days 8 to 14). On Day 15, these participants will enter the maintenance period, receiving a 60 mg QAM dose through the end of the Intervention Period (Day 84). No dosage adjustments will be allowed.

Participants who complete Study 1 PD can rollover into the ongoing Study 3 LTSS with Day 84 assessments carried forth from Study 1 PD.

### Study 2 RW

All participants assigned to Study 2 RW, who continue to meet all clinical trial entry criteria on Day 1, will enter the RW Lead-in Period.

### RW Lead-In Period

During the RW Lead-in Period participants will be administered Compound 1 during the Home Health Visit on Day 1 and self-administered from Day 2 through Day 56. These participants will be blinded to whether they are participating in this study or Study 1 PD and will therefore be administered Compound 1 in a DB fashion.

On Day 1 (Baseline) participants will complete CGI-S, PGI-S, and Archimedes Spiral assessments in that order before dosing.

All participants will undergo a titration period that will consist of a 20 mg QAM dose during Week 1 (Days 1 to 7) and a 40 mg QAM dose during Week 2 (Days 8 to 14). On Day 15, these participants will enter the maintenance period, receiving a 60 mg QAM dose through the end of the Intervention Period (Day 56). No dosage adjustments will be allowed.

### RW Period

Based on pre-defined selection criteria, a subset of participants who are responders to Compound 1 will be randomized 1:1 to either continue Compound 1 or receive placebo in a DB fashion. No Compound 1 taper is required prior to starting placebo. The participants who do not meet these criteria will remain on Compound 1 at 60 mg throughout the remainder of the study. Study visits will be conducted weekly, throughout the duration of the RW period as outlined in the SoA.

Participants who complete Study 2 RW can rollover into the ongoing Study 3 LTSS with Day 84 assessments carried forth from Study 2 PD.

Participants who meet the criteria of worsening tremor (according to the predefined relapse criterion) during the RW, will rollover to Study 3 LTSS. The final assessments conducted in Study 2 RW will carry forth to Study 3 LTSS Day 84.

### Study 3 LTSS

Participants on placebo in Studies 1 PD or 2 RW will undergo a DB titration period that will consist of a 20 mg QAM dose during the first week (Days 84 to 90) and a 40 mg QAM dose during the second week (Days 91 to 97) (Table 9). Participants who were receiving 60 mg Compound 1 in a pivotal study when rolling over to the LTSS will remain on the 60 mg dose throughout the titration period in a blinded fashion. On Day 15, all participants will enter the open-label maintenance period, receiving a 60 mg QAM dose through the remainder of the trial. Participants in the Phase 2 OLE (Example 2) will rollover to Study 3 LTSS at the 60 mg dose or titrate over a 2-week period to 60 mg if appropriate based on their current dose. No dosage adjustments will be allowed in Study 3 LTSS.

### Scientific Rationale for Clinical Trial Design

This Protocol includes 2 simultaneous pivotal DB studies designed to further assess the favorable efficacy and safety profiles of Compound I demonstrated throughout the Phase 2 program. A multi-study protocol offers multiple benefits over a traditional single trial protocol. Shared trial infrastructure allows more efficiency in enrollment, operations, data collection and management as well as overall resource utilization across the 3 studies. Furthermore, simultaneous study enrollment ensures a more homogeneous patient population across the two pivotal Phase 3 trials (Study 1 PD and Study 2 RW). By initiating Study 1 PD and Study 2 RW simultaneously under one protocol, administration of Compound 1 treatment in the RW Lead-in Period of Study 2 RW will be blinded, resulting in a more accurate determination of Compound 1 responders. Finally, all Study 1 PD and Study 2 RW completers can directly enter the LTSS, allowing seamless transition from a participant perspective and continuous safety data collection from a sponsor perspective.

Study 1 PD and Study 2 RW will be conducted using 2 different study designs. Study 1 PD is similar to the Phase 2 trial described in Example 1, but extends the total treatment period to 12 weeks to allow assessment of chronic treatment. The Study 2 RW design was chosen for multiple reasons. First, durability of effect will be further confirmed in the RW arm designed to assess a subpopulation defined as ulixacaltamide responders. The RW design is expected to be useful since ET is known to be heterogeneous; and given this heterogeneity (i.e., unique patient characteristics, genotype/etiology or phenotype), it is likely that there are subpopulations of ET participants that will respond more favorably to treatment than other subpopulations. The RW design identifies this subpopulation, their response to treatment, and will therefore lead to a better understanding of the responder population. In addition, participants will be assessed weekly during the RW period, and those who meet the predefined relapse criterion will rollover into Study 3 LTSS. This limits the duration of placebo for those that are true responders.

The Intervention Periods in Studies 1 PD and 2 RW are up to 12 weeks in duration, including the 2-week titration. A 2 week titration period is considered appropriate based on the tolerability observed in Phase 2. Additionally, a 10-week maintenance treatment period at the 60 mg dose is considered sufficient to establish the durability of the effect of chronic dosing. This is supported by evidence of a treatment effect as early as 2 weeks in the Phase 2 trials. In Phase 2, the LSM mADL11 score change from baseline showed a treatment effect relative to placebo of 1.44 on Day 28, with sustained effect throughout the remainder of the study (Day 56). A favorable treatment effect of 1.03 points in mADLII relative to placebo was observed as early as the first assessment point (Day 14) in participants without intention tremor (IT).

In the Phase 2 OLE study (Example 2), the requirement for rescue treatment in the placebo group was observed within the 3-week crossover period suggesting a rapid washout out of drug effect and further supporting the 4-week RW period chosen for Study 2 RW.

The presence of IT is of special interest because it is a distinct type of action tremor observed in some individuals with ET that can be associated with other unique neurologic conditions. IT is characterized by a high amplitude, low frequency coarse tremor that worsens with movement toward a target as opposed to the action or postural tremor otherwise observed in ET in which tremor frequency and amplitude is more consistent throughout the trajectory of movement. Because IT can be associated with a diagnosis other than ET, participants with IT will be assessed during screening to determine if there is a potential cerebellar etiology different than that of ET. Only participants determined by the Eligibility Review Committee (ERC) central reviewer to have IT associated with ET (ET with IT) and not an alternative etiology for IT will be enrolled in the study. Functional, physiologic and anatomical data suggests that ET with IT may represent a unique clinical manifestation that can create a potentially confounding effect in terms of clinical study outcome measures based on functional activities. Therefore, this subpopulation could confound the measurement and interpretation of the primary outcome measure, ADL performance. Based on this, participants with IT will be enrolled only if IT is determined to be associated with ET and not an alternative etiology. Participants assigned to Studies 1 PD or 2 RW will be stratified for the presence of ET with IT. Participants determined to have ET with IT will be included in Study 1 PD and Study 2 RW sensitivity analyses.

The primary endpoints for Study 1PD and Study 2 RW are based on mADL11 (TETRAS-ADL items 1-11 with transformed score 0-3). In the Phase 2 trial described in Example 1, the primary endpoint was mADL (TETRAS-ADL items 1-11+performance subscale [PS] items 6 and 7 with transformed score 0-3). While mADL score was reduced in the Phase 2 studies, careful analysis of the data from the Phase 2 trial described in Example 1 revealed that the PS items (Archimedes spirals and cursive handwriting) and ADL item 12 (social impact) did not have sufficient sensitivity, reliability, specificity, and/or clinical meaningfulness to assess ET severity. Thus, in alignment with feedback from the FDA, the mADL11 score was chosen as the primary efficacy endpoint for Study 1 PD and as a secondary endpoint for Study 2 RW. Dominant hand Archimedes spiral will be analyzed as a secondary endpoint.

This is a decentralized clinical trial. The decreased burden of a decentralized trial would enhance involvement of participants without access to research clinics, participation of older participants that are limited by transportation (and other issues, e.g., mobility, etc.), and younger participants who are potentially still working and need a more convenient clinical trial schedule. This will improve trial participant engagement, recruitment, enrollment, and retention not only of a meaningfully diverse clinical population but also can increase investigator diversity which is important for clinical trials in the US. Furthermore, home-based collection of efficacy assessments is ideal since study assessments are functionally relevant ADLs that focus on participants daily experience and disability associated with ET. The Phase 2 crossover study shows that a decentralized trial can be effectively and safely performed for participants with moderate to severe ET, and the TETRAS-ADL can be assessed via telehealth with consistency in mADL11 observed over multiple consecutive timepoints. Given this, these assessments are feasible to collect, and bias can be reduced through collection by a decentralized investigator. Finally, a decentralized trial is appropriate given the well-tolerated and well-characterized safety profile of Compound 1 as noted in the Phase 2 study as described in Example 1. Compound 1 has stable characteristics that allow direct-to-patient shipping, simple drug administration, and simple safety monitoring appropriate for a decentralized trial.

### End of Clinical Trial Definition

A participant is considered to have completed the Studies 1 PD and 2 RW when they either rollover to Study 3 LTSS or complete a SFU or ED visit. The end of Study 3 LTSS will occur when participants have completed the last planned assessment, as shown in SoA. The trial may be terminated at any time by the sponsor.

### Justification for Dose

Ulixacaltamide, when administered as the modified release (MR) formulation, in the context of titration, has demonstrated a favorable safety profile in healthy participants and patients with ET throughout the Phase 2 development program at doses ranging from 5 to 120 mg for up to 129 days.

In the Phase 2 trial described in Example 1, participants were randomized to placebo or 60 or 100 mg of Compound 1. When evaluating the change from baseline versus placebo on the mADL11 endpoint, the magnitude of benefit was largely similar between participants in the 60 mg or 100 mg Compound 1 groups. There was no additional benefit observed by increasing the dose to 100 mg and no significant difference in the safety profile between the 60 mg and 100 mg groups. Thus, the benefit: risk ratio for the 60 mg dose is ideal without the need to evaluate additional maintenance doses lower than 60 mg.

The effect of a high-fat, high-calorie meal on the pharmacokinetics of Compound I was evaluated in healthy participants who had previously received Compound 1 under fasted conditions in a Phase 1 trial. The effect of a high-fat, high-calorie meal on the PK of Compound 1 was evaluated at the 120 mg dose level after healthy adult participants had titrated to reach steady-state exposure. Following administration of 120 mg dose in the fed state or fasted state, the maximum plasma drug concentration (Cₘₐₓ) and AUC, values were similar. Based on this analysis, Compound 1 may be administered without regard to food.

### Study Drug Administration

Compound 1 will be supplied as 20 mg, 40 mg, and 60 mg modified-release tablets. Matching placebo will also be supplied. Study drug is administered orally QAM with or without food and will be provided to participants in pre-packaged bottles. Participants receiving ulixacaltamide will receive 1 tablet of study drug per day. Participants in the placebo group will receive one tablet of matching placebo per day. For each home health/telehealth visit, the dose must be taken from the previously dispensed bottle. The interactive response technology (IRT) system can dispense replacement bottles, if necessary.

Eligible participants will be randomized to receive oral Compound 1 or placebo daily. Fixed titration regimens will be used for the 3 studies beginning with 20 mg for 7 days, then 40 mg for 7 days followed by 60 mg maintenance dose (note that participants dosed with placebo in Study 1 PD and Study 2 RW will require blinded titration upon rollover into Study 3 LTSS). Dose adjustments will not be allowed.

### Efficacy Assessments

All TETRAS-ADL assessments throughout the study will be conducted by a trained and qualified investigator. The Baseline CGI-S, PGI-S, and Archimedes Spiral assessments on Day 1 (Baseline) must be conducted pre-dose.

### The Essential Tremor Rating Assessment Scale

The TETRAS was developed by the Tremor Research Group (TRG; www.tremorresearchgroup.org) to quantify ET severity and its impact on ADLs. The full scale has 2 sections, the Performance and ADL subscales. This study will collect only the TETRAS-ADL.

### TETRAS Activities of Daily Living

The TETRAS-ADL subscale is a 12-item assessment of typical daily activities that are impacted by tremor. Activities are assessed in the following functional domains: speaking, feeding, drinking, personal hygiene, dressing, pouring, carrying, using keys, writing, working, overall disability and social activity. The impact to each function is rated on a 5-point Likert scale from 0 to 4. The full ADL subscale score is calculated as the sum of all 12 items and ranges from 0 to 48.

### TETRAS Modified Activities of Daily Living 11

The mADL11 score is a composite sum of items 1 to 11 (omission of item 12 social impact score) of the TETRAS-ADL subscale. Scoring of each item is transformed to a 4-point scale from 0 to 3 with total score range from 0 to 33. The TETRAS ADL will be collected and the mADL11 will be used for data analysis purposes.

### Clinical Global Impression

The Clinical Global Impressions (CGI) scales are commonly used measures of symptom severity and response in clinical trials (Guy 1976). The CGI-S scale will be used in this clinical trial.

### Clinical Global Impression-Severity

The CGI-S assesses the clinician's impression of the participant's current illness state. The clinician should use his or her total clinical experience with this patient population and rate the current severity of the participant's ET on a 7-point scale from 1 (normal, not at all ill) to 7 (among the most extremely ill patients).

### Patient Global Impression

### Patient Global Impression-Change

The PGI-C assesses the participant's improvement (or worsening). The participant is required to assess his or her condition relative to Baseline (Pre-dose on Day 1) on a 7-point scale from 1 (very much improved) to 7 (very much worse). In all cases, the assessment should be made independent of whether the participant believes the improvement/worsening is drug-related or not.

### Patient Global Impression-Severity

The PGI-S assesses the participant's impression of his or her current illness state. The participant is required to assess his or her condition on a 7-point scale from 1 (Not present) to 7 (extremely severe).

### Archimedes Spiral

Participants will draw Archimedes Spirals with their dominant hand on an unlined secured standard size (letter) paper, which includes preformed spiral drawings. A specific pen (to standardize across all drawings) will be distributed for use. Participants will be instructed to draw a spiral without crossing lines in the preformed spiral template using their dominant hand, without practice, and without leaning on the dominant forearm, wrist or hand. Descriptive analyses will be used to summarize the findings.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any clement(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating essential tremor, said method comprising administering to a subject in need thereof compound of formula (I): 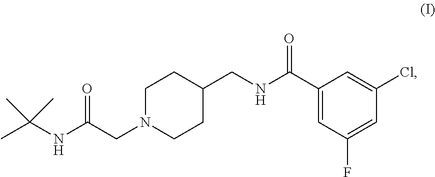 [SMILES: CC(C)(C)NC(=O)CN1CCC(CNC(=O)c2cc(F)cc(Cl)c2)CC1]
or a pharmaceutically acceptable salt thereof,
wherein the compound of formula (I), or a pharmaceutically acceptable salt thereof, is administered to the subject once daily at a dose of about 60 mg to about 100 mg;
such that The Essential Tremor Rating Assessment Scale (TETRAS) performance score of said subject is decreased following administration of the compound of formula (I), or a pharmaceutically acceptable salt thereof, as compared to the TETRAS performance score of said subject prior to the administration of the compound of formula (I), or a pharmaceutically acceptable salt thereof.

2. The method of claim 1, wherein the compound of Formula (I), or a pharmaceutically acceptable salt thereof, is administered to said subject at a dose of about 60 mg.

3. The method of claim 1, wherein the compound of formula (I), or a pharmaceutically acceptable salt thereof, is administered to said subject at a dose of about 100 mg.

4. The method of any one of claims 1-3, wherein the compound of formula (I), or a pharmaceutically acceptable salt thereof, is administered to said subject following an initial titration period.

5. The method of claim 4, wherein the compound of formula (I), or a pharmaceutically acceptable salt thereof, is administered to said subject at a dose of about 60 mg following an initial titration period;
wherein said initial titration period comprises administering to the subject the compound of formula (I), or a pharmaceutically acceptable salt thereof, once daily at a dose of about 5 mg during a first time period; followed by a dose of about 10 mg during a second time period; followed by a dose of about 20 mg during a third time period; followed by a dose of about 40 mg during a fourth time period.

6. The method of claim 5, wherein the first time period, the second time period, the third time period and the fourth time period are each 7 days long.

7. The method of claim 4, wherein the compound of formula (I), or a pharmaceutically acceptable salt thereof, is administered to said subject at a dose of about 100 mg following an initial titration period;
wherein said initial titration period comprises administering to the subject the compound of formula (I), or a pharmaceutically acceptable salt thereof, once daily at a dose of about 5 mg during a first time period; followed by a dose of about 10 mg during a second time period; followed by a dose of about 20 mg during a third time period; followed by a dose of about 40 mg during a fourth time period; followed by a dose of about 60 mg during a fifth time period, followed by a dose of about 80 mg during a sixth time period.

8. The method of claim 7, wherein the first time period, the second time period, the third time period, the fourth time period, the fifth time period and the sixth time period are each 7 days long.

9. The method of any one of claims 1-8, wherein the compound of formula (I), or a pharmaceutically acceptable salt thereof, is administered to the subject for at least 8 weeks.

10. The method of any one of claims 1-8, wherein the compound of formula (I), or a pharmaceutically acceptable salt thereof, is administered to the subject for at least 12 weeks.

11. The method of any one of claims 1-8, wherein the compound of formula (I), or a pharmaceutically acceptable salt thereof, is administered to the subject for at least 14 weeks.

12. The method of any one of claims 1-8, wherein the compound of formula (I), or a pharmaceutically acceptable salt thereof, is administered to the subject for at least 52 weeks.

13. The method of any one of claims 1-12, wherein the TETRAS performance score comprises ADL subscore, and wherein the ADL subscore is decreased by at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points following administration of the compound of formula (I), as compared to the ADL subscore of said subject prior to the administration of the compound of formula (I).

14. The method of claim 13, wherein the ADL subscore is decreased by at least about 2 points following at least 8 weeks of administration of the compound of formula (I), or a pharmaceutically acceptable salt thereof.

15. The method of claim 13, wherein the ADL subscore is decreased by at least about 3 points following at least 8 weeks of administration of the compound of formula (I), or a pharmaceutically acceptable salt thereof.

16. The method of any one of claims 1-12, wherein the TETRAS performance score comprises mADL subscore, and wherein the mADL subscore is decreased by at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points following administration of the compound of formula (I), as compared to the ADL subscore of said subject prior to the administration of the compound of formula (I).

17. The method of claim 16, wherein the mADL subscore is decreased by at least about 2 points following at least 8 weeks of administration of the compound of formula (I), or a pharmaceutically acceptable salt thereof.

18. The method of claim 17, wherein the mADL subscore is decreased by at least about 3 points following at least 8 weeks of administration of the compound of formula (I), or a pharmaceutically acceptable salt thereof.

19. The method of claim 16, wherein the mADL subscore is decreased by at least about 4 points following at least 8 weeks of administration of the compound of formula (I), or a pharmaceutically acceptable salt thereof.

20. The method of any one of claims 16-19, wherein a subscore for at least one item assessed as a part of mADL subscale is decreased, wherein said item is selected from the group consisting of: item 3 (drinking from a glass); item 4 (hygiene); item 5 (dressing); item 6 (pouring); item 7 (carrying); item 8 (using keys); item 9 (writing); and item 11 (overall disability).

21. The method of any one of claims 1-12, wherein the TETRAS performance score comprises mADL11 subscore, and wherein the mADL11 subscore is decreased by at least about 0.5 points, at least about 1 point, at least about 2 points, at least about 3 points, at least about 4 points, at least about 5 points, at least about 6 points, at least about 7 points, at least about 8 points, at least about 9 points or at least about 10 points following administration of the compound of formula (I), as compared to the ADL subscore of said subject prior to the administration of the compound of formula (I).

22. The method of claim 19, wherein the mADL11 subscore is decreased by at least about 2 points following at least 8 weeks of administration of the compound of formula (I), or a pharmaceutically acceptable salt thereof.

23. The method of claim 19, wherein the mADL11 subscore is decreased by at least about 2.5 points following at least 8 weeks of administration of the compound of formula (I), or a pharmaceutically acceptable salt thereof.

24. The method of any one of claims 1-23, wherein said subject does not have intention tremor.

25. The method of any one of claims 1-24, wherein said subject is a human.
